(19) United States
(12) Patent Application Publication
(10) Pub. No.: US 2019/0161731 A1
(43) Pub. Date: May 30, 2019

(54) DIRECT REPROGRAMMING OF SOMATIC CELLS INTO MYOGENIC CELLS

(71) Applicant: THE GENERAL HOSPITAL CORPORATION, Boston, MA (US)

(72) Inventors: Konrad HOCHEDLINGER, Boston, MA (US); Ori BAR-NUR, Boston, MA (US)

(73) Assignee: THE GENERAL HOSPITAL CORPORATION, Boston, MA (US)

(21) Appl. No.: 16/091,700

(22) PCT Filed: Apr. 6, 2017
(86) PCT No.: PCT/US17/26421

§ 371 Date: Oct. 5, 2018

Related U.S. Application Data
(60) Provisional application No. 62/318,885, filed on Apr. 6, 2016.

(51) Int. Cl. C12N 5/077; A61K 35/34

(52) U.S. Cl. CPC C12N 5/0652; A61K 35/34; C12N 2500/32; C12N 2501/115; C12N 2501/01; C12N 2501/999; C12N 2500/38

(57) ABSTRACT

Described herein are methods of generating induced muscle progenitor cells (iMPCs) and uses thereof. Embodiments further provide for methods of promoting muscle regeneration and/or repair and methods of treating a muscle disease or disorder.

### CROSS-REFERENCE TO RELATED APPLICATIONS

This application claims benefit under 35 U.S.C. § 119(e) of U.S. Provisional Application No. 62/318,885 filed Apr. 6, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 6, 2017, is named 030258-089071_SL.txt and is 2,605 bytes in size.

### FIELD OF THE INVENTION

The invention relates to a method of preparing and using skeletal muscle progenitors.

### BACKGROUND

Skeletal muscle is largely comprised of differentiated, polynucleated myofibers responsible for contraction and thus movement. In addition, muscle tissue contains a quiescent population of mononucleated stem cells termed satellite cells, which are located between the basal lamina and sarcolemma of myofibers. Satellite cells are maintained in a quiescent state under homeostatic conditions but undergo activation following tissue injury. Once activated, satellite cells generate transit-amplifying progenitors termed myoblasts, which then differentiate and fuse with one another or with resident myofibers to regenerate damaged tissue. Remarkably, individual satellite cells have the potential to produce myofibers and replenish the satellite cell niche when transplanted into damaged muscle, documenting their self-renewal and differentiation potential.

### SUMMARY

The following embodiments and aspects thereof are described and illustrated in conjunction with systems, compositions and methods which are meant to be exemplary and illustrative, not limiting in scope.

Various embodiments of the present invention provide for a method of generating induced muscle progenitor cells (iMPCs), the method comprising: treating a population of somatic cells obtained from a subject with cyclic AMP agonist, and a TGF-β inhibitor for a time and under conditions that induce dedifferentiation of the somatic cells to a population of cells comprising iMPCs.

In one embodiment, the somatic cells can be fibroblasts.

In various embodiments, the cyclic AMP agonist is forskolin. In other embodiments, the TGF-β inhibitor is RepSox, SB-431542 or ALK5 Inhibitor II. In various embodiments, the TGF-β inhibitor is RepSox.

In various embodiments, the method further comprises expressing an exogenous myogenic factor in the somatic cells. In some embodiments, the exogenous myogenic factor is MyoD. In other embodiments, the exogenous MyoD is expressed transiently. In yet other embodiments, the exogenous MyoD is expressed for a minimum of 2 days.

In various embodiments, the somatic cells are cells isolated or derived from a muscle biopsy or muscle-derived explant sample and the iMPCs are muscle-induced iMPCs (M-iMPCs).

In various embodiments, the method further comprises culturing the somatic cells and/or population of cells comprising iMPCs with ascorbic acid.

In various embodiments, the method further comprises a step of isolating an iMPC and plating it as a clonal culture. In various other embodiments, the iMPCs are proliferative, self-renewing and capable of forming skeletal muscle myotubes. In some embodiments, the iMPCs can be maintained in culture for at least 4 months. In yet other embodiments, the iMPCs can be maintained in culture for at least 6 months or more.

In various embodiments, the population of cells is a heterogeneous population of cultured cells. In some embodiments, the population of cells further comprises differentiated skeletal muscle cells. Such differentiated skeletal muscle cells can arise, for example, from iMPCs or M-iMPCs.

In other embodiments, the dedifferentiation of the somatic cells to iMPCs does not go through a transient pluripotent state. In yet other embodiments, the population expresses one or more of the following markers: Pax7, Myf5, Cxcr4, Myf6, VCAM1, Myog and MyHC. In various other embodiments, the iMPCs do not detectably express fibroblast markers. In some embodiments, the fibroblast markers are Col5a1, Thy1, and Fbln5. In other embodiments, the iMPCs are mononucleated.

Various embodiments of the present invention also provide for an in vitro heterogeneous population of skeletal muscle cells comprising induced muscle progenitor cells (iMPCs). In various embodiments, the heterogeneous population can be maintained in culture without loss of phenotype for at least 6 months. In various other embodiments, the in vitro heterogeneous population further comprises medium comprising ascorbic acid, GSK3 inhibitor and FGF (e.g., bFGF).

Various embodiments of the present invention also provide for a method for promoting muscle regeneration and/or repair, the method comprising: administering a therapeutically effective amount of iMPCs to a subject in need thereof. In various embodiments, the iMPCs are prepared according to the methods described herein. In various other embodiments, the iMPCs are autologous to the subject. In yet other embodiments, the therapeutically effective amount comprises at least 1×10⁵ cells. In other embodiments, the therapeutically effective amount comprises at least 1×10⁶ cells. In other embodiments, the therapeutically effective amount comprises at least 5×10⁶, at least 1×10⁷, at least 5×10⁷, at least 1×10⁸, at least 5×10⁸, at least 1×10⁹ or more cells.

Various embodiments of the present invention also provide for a method for treating a muscle disease or disorder, the method comprising: administering a therapeutically effective amount of iMPCs to a subject in need thereof. In various embodiments, the iMPCs are prepared according to the methods described herein. In various embodiments, the iMPCs are autologous to the subject. In various other embodiments, the therapeutically effective amount comprises at least 1×10⁵ cells. In some embodiments, the therapeutically effective amount comprises at least 1×10⁶ cells. In yet other embodiments, the muscle disease or disorder is characterized by a gene mutation and/or deficiency. Provided herein are methods and systems for modeling muscle disease, comprising generating iMPCs from an individual with a muscle disease.

Various embodiments of the present invention provide for a method of screening for a drug useful in the treatment of a disease comprising obtaining a sample from a subject with the disease; generating iMPCs by the methods disclosed herein; contacting the iMPCs generated with a drug, and determining the effect of the drug on the iMPC cells.

In various embodiments, the disease is characterized by a gene mutation and/or deficiency. In various other embodiments, the disease is a muscle-associated disorder. In yet other embodiments, the muscle-associated disorder is Duchenne's muscular dystrophy, Becker muscular dystrophy, facioscapulohumeral muscular dystrophy, myotonic dystrophy, congenital muscular dystrophy, distal muscular dystrophy, emery-dreifuss muscular dystrophy, oculopharyngeal muscular dystrophy, or limb girdle muscular dystrophy.

In various embodiments, the drug is a known or experimental drug. In other embodiments, a combination of drugs can be screened. In various embodiments, the drug is beneficial if an increase in the mutated gene's expression is observed and the drug is not beneficial if a decrease or no change in the mutated gene's expression is observed relative to a reference value. In various other embodiments, the drug is beneficial if there is an increase in muscle regeneration and/or repair and the drug is not beneficial if there is a decrease or no change in muscle regeneration and/or repair.

In various embodiments, the method further comprises administering a drug thus screened that has been determined to be beneficial to the subject with the disease. In various embodiments, the subject has, is diagnosed as having or at risk of developing a muscle-associated disease.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1F depict in accordance with various embodiments of the invention, that ascorbic acid and GSK3β inhibitor (“AGi”) facilitate the conversion of MEFs to postmitotic skeletal muscle cells upon overexpression of MyoD. FIG. 1A) Schematic of experimental design.

FIG. 1B) Quantitative PCR analysis for expression of the skeletal muscle differentiation marker myosin heavy chain (MyHC) in the indicated samples. The myoblast cell lines C2C12 and cells differentiated from it (C2C12-diff) were used as negative and positive controls, respectively (n=3 biological replicates; error bars, s.d.; for C2C12 and C2C12− diff technical replicates are shown). FIG. 1C) Representative bright-field images of cells overexpressing MyoD or MyoD+AGi for 4 days. White arrowheads indicate polynucleated myotubes. Scale bars, 500μM. FIG. 1D) Representative Immunofluorescence images showing staining for MyHC in the indicated treated samples. Scale bars, 100μM. Autofluor., autofluorescence control. FIG. 1E) Graph showing quantification of the number of MyHC positive cells in the indicated samples. 27 random fields were chosen for each biological replicate. For each replicate 1*10⁵ cells were used (n=3 biological replicates; error bars, s.d.). FIG. 1F) A graph showing the ratio of mononucleated vs. polynucleated MyHC positive cells in the indicated samples. Polynucleated cells are indicative of mature, fused muscle fibers. 27 random fields were chosen for each biological replicate (n=3 biological replicates; error bars, s.d.).

FIGS. 2A-2E depict in accordance with various embodiments of the invention, small molecule treatment in combination with MyoD overexpression endows MEFs with a proliferative muscle progenitor-like state. FIG. 2A) Experimental design assessing if small molecules can endow MEFs with a proliferative muscle progenitor-like state upon MyoD overexpression. FIG. 2B) Representative bright-field images of cells induced to overexpress MyoD in the presence of the indicated small molecules. A proliferative, contractile cell population was generated only in the presence of the cyclic AMP agonist Forskolin (F), the ALK5 inhibitor RepSox (R) and ascorbic acid (AA). This experiment was validated using three different MEF lines; for each replicate 1*10⁵ cells were used per treatment. FIG. 2C) Table depicting the quantitative conversion efficiency of MEFs into induced muscle progenitor-like cells upon MyoD overexpression and exposure to the indicated small molecules. Serum replacement media can be replaced with ascorbic acid. FIG. 2D) Experimental design assessing if the generation of a muscle progenitor-like cell population is dependent on the duration of MyoD overexpression and the presence of Forskolin and RepSox (FR). FIG. 2E) Table showing the temporal requirement for MyoD expression to generate muscle progenitor-like cells in the presence of Forkolin, RepSox and serum replacement media. Doxycycline and FR were applied for the indicated length of time. Following dox withdrawal, cells were propagated in the presence or absence of FR and scored for proliferation and contractility 7 days after the last time point (12 days). This experiment was validated using three different MEF lines, for each replicate 1*10⁵ cells were used per time point. For all subsequent figures, induced muscle progenitor-like cells are referred to as induced muscle progenitor cells (iMPCs) for simplicity.

FIGS. 3A-3E depict in accordance with various embodiments of the invention, the molecular characterization of induced muscle progenitor cells (iMPCs) generated by MyoD overexpression in the presence of Forskolin and RepSox (FR). FIG. 3A) Representative images of iMPCs. Note spheroid structures connected by elongated skeletal myotubes. FIG. 3B) Quantitative PCR analysis for muscle-specific genes in the indicated cell lines. Shown are results from three MEF lines (MEFs), 2 bulk iMPC cultures and 4 iMPC clones that were derived from single iMPC clusters and propagated for 5-10 passages. The myoblast progenitor cell line C2C12 was used as control (error bars, S.D.; *P<0.05). **P<0.005, ***P<0.0005). FIG. 3C) Representative immunofluorescence images showing staining for skeletal muscle-specific genes in iMPC clone#3 (passage 8). MyHC expressing cells are predominately polynucleated while Pax7 expressing cells are mononucleated. Scale bars, 50μM. Autofluor., autofluorescence control. FIG. 3D) Quantitative PCR analysis for skeletal muscle-specific genes in early (p6) and late (p20) passage iMPC clone. MEFs served as a negative control (N=3 independent replicates; error bars S.D.). FIG. 3E) Representative immunofluorescence images show staining for skeletal muscle-related genes in late-passage iMPC clone (passage 20). Scale bars, 50μM. Autofluor., autofluorescence control.

FIGS. 4A-4F depict in accordance with various embodiments of the invention, global transcriptome analysis of iMPCs which shows similarities to muscle-derived satellite cells. FIG. 4A) Expression of pluripotency-, MEF-, muscle-, and cardiac-associated markers in the indicated samples. ASCs., activated satellite cells, representing a proliferative type of adult skeletal muscle-derived stem cells. C2C12 cells were used as a control for myoblast progenitor cells. There is a lack of pluripotency, MEF or cardiac-related genes in the established iMPC clone and in MEFs exposed to MyoD+FR. FIG. 4B) Graphs showing the top upregulated genes by expression microarray in iMPCs compared to MEFs (top) and in bulk MEF cultures exposed to MyoD+FR in comparison to MEFs exposed to MyoD alone (bottom). FIG. 4C) Dendrogram analysis based on gene expression microarrays for indicated samples. FIG. 4D) Functional annotation analysis calculated by DAVID for upregulated genes (2-fold or more) in iMPC#3 vs. MEFs. Benjamini-Hochberg (BH) adjusted P values are presented. Top categories are shown together with the number of genes. FIG. 4E) Scatter plot analysis of linear regression coefficient (R²) for global gene expression between the indicated samples. FIG. 4F) Venn-diagram analysis showing the overlap of upregulated genes (>2-fold) between quiescent stem cells (QSCs), representing a quiescent satellite cells, activated stem cells (ASCs), representing activated satellite cells/myoblasts, and iMPC#3 in comparison to MEFs. Overlap of satellite-related genes in all three samples and the overlap of differentiation-related skeletal muscle genes between iMPCs and ASCs, but not QSCs are depicted.

FIGS. 5A-5H depict in accordance with various embodiments of the invention, that iMPCs originate from fibroblasts and do not pass through a transient pluripotent (Oct4⁺) state. FIG. 5A) Experimental design assessing if iMPC are derived from fibroblasts (Thy1⁺) or other contaminating cell types (Thy1⁻). FIG. 5B) Quantification of contracting colonies in sorted Thy⁺ fibroblasts expressing MyoD or MyoD+FR. For each replicate 1*10⁵ cells were used (n=3 biological replicates, 2 MEF lines and 1 tail tip fibroblast/TTF line were used; error bars, s.d. ***P<0.0005). FIG. 5C) Representative Immunofluorescence images for MyHC and Pax7 expression in sorted Thy1⁺ fibroblasts expressing either MyoD or MyoD+FR for 14 days. Scale bars, 100μM. Autof, autofluorescence control. FIG. 5D) Quantification of Pax7 positive nuclei in 3 random fields taken from sorted Thy1⁺ cell populations expressing either MyoD or MyoD+FR for 14 days. For each replicate 1*10⁵ cells were used (n=3 independent replicates; error bars, s.d. **P<0.005). FIG. 5E) Experimental design assessing if iMPC formation requires passage through an Oct4⁺ pluripotent state. No iMPC colonies are expected to form if MEFs pass through a transient Oct4⁺ state, which would result in activation of the DTA suicide gene. FIG. 5F) Quantification of contracting colonies generated with and without 4-OHT from Oct4-CreER×Rosa26-LSL-DTA MEFs. For each replicate 1*10⁵ cells were used (n=3 independent replicates; error bars, s.d.). FIG. 5G) Representative immunofluorescence images show staining for Pax7 in iMPCs derived from Oct4-CreER×Rosa26-LSL-DTA MEFs with and without 4-OHT. FIG. 5H) Quantification of Pax7 positive nuclei in 3 random fields taken from Oct4-CreER×Rosa26-LSL-DTA MEFs treated with MyoD+FR with and without 4-OHT. For each replicate 1*10⁵ cells were used (n=3 independent replicates; error bars, s.d.).

FIGS. 6A-6H depict in accordance with various embodiments of the invention, iMPC cultures containing Pax7⁺ satellite-like cells that produce muscle fibers. FIG. 6A) Schematic of lineage tracing approach to assess if iMPCs pass through a Pax7⁺ satellite cell-like state. FIG. 6B) Flow cytometry analysis of Pax7-CreER×Rosa26-LSL-EYFP MEFs treated with and without 4-OHT for 24 hours. The absence of EYFP signal indicates that MEFs do not contain contaminating satellite cells and that the CreER system is not leaky. FIG. 6C) Generation of EYFP⁺ iMPCs from Pax7-CreER×Rosa26-LSL-EYFP MEFs. Shown are representative images of iMPC colonies generated upon exposure to MyoD or MyoD+FR with and without 4-OHT. EYFP signal detected in the MyoD+FR+4-OHT condition. Autofluor., autofluorescence control. FIG. 6D) Flow cytometry analysis and specificity of the Pax7-CreER×Rosa26-LSL-EYFP system. Shown are treated Pax7-CreER×Rosa26-LSL-EYFP MEFs after 6 days of treatment as indicated. Only with FR+4-OHT treatment EYFP positive cells are detected. FIG. 6E) Generation of EYFP⁺ iMPCs from Pax7-CreER×Rosa26-LSL-EYFP tail tip fibroblasts/TTFs, representing a type of adult fibroblasts. Shown are representative images of iMPC colonies generated using MyoD+4-OHT or MyoD+FR exposure with and without 4-OHT treatment. Autofluor., autofluorescence control. FIG. 6F) Flow cytometry analysis of Pax7-CreER×Rosa26-LSL-EYFP iMPCs derived from TTFs using the indicated conditions. Only with FR+4-OHT treatment are Pax7 positive cells detected. FIG. 6G) Flow cytometry analysis of iMPC clone for the indicated surface makers. FIG. 6H) Flow cytometry analysis of Pax7-CreER×Rosa26-LSL-EYFP MEFs exposed to MyoD+F/R conditions with and without 4-OHT and analyzed at indicated passages. iMPCs at higher passage have increased labeling. Representative result of two independent biological replicates. The PE-Cy7 channel was used to control for autofluorescence.

FIG. 7 depicts in accordance with various embodiments of the invention, a doxycycline-dependent lentiviral system to induce MyoD expression in fibroblasts. Representative Immunofluorescence images show staining for MyoD in indicated samples following 24 hours of doxycycline administration. MEFs were infected with lentiviral vectors expressing the reverse tetracycline transactivator (rtTA) and the tetOP-MyoD gene, respectively. Doxycycline was added for 24 hours, followed by staining for MyoD expression. Untreated cells served as controls. Scale bars, 100 μM. Autof., autofluorescence control using the green (GFP) channel.

FIGS. 8A-8D depict in accordance with various embodiments of the invention, that combined MyoD expression and small molecule treatment in MEFs gives rise to a proliferative, skeletal muscle progenitor like cell population. FIG. 8A) Representative Immunofluorescence images of MyHC (green) and Pax7 (red) expression in MEFs expressing MyoD in the presence of Forskolin (F) and RepSox (R) and in medium containing fetal calf serum (FCS), serum replacement (SR) and basic-FGF (bFGF). Pax7⁺ cells were detected only in the presence of FR. Scale bars, 50 μM. Autofluor., autofluorescence control. FIG. 8B) Ascorbic acid is critical for the generation of iMPCs. Cells are generally reprogrammed in iMPC medium, which contain fetal calf serum (FCS) and serum replacement (SR). A key component of SR media is ascorbic acid. Withdrawal of SR prevents iMPC formation, as indicated by the lack of Pax7 positivity, while addition of ascorbic acid partially rescues this phenotype and leads to the generation Pax7⁺ iMPCs. Scale bars, 50μM. Autofluor., autofluorescence control. FIG. 8C) Representative Immunofluorescence images of cells overexpressing MyoD in the presence of the indicated small molecules. GSK3β inhibitor induces a Pax7⁺ population only in the presence of FR. FIG. 8D) Representative Immunofluorescence images of cells exposed to iMPC medium+FR without MyoD overexpression and assessment of Pax7 positivity. Pax7⁺ iMPCs emerge even without MyoD expression, albeit at extremely low efficiency and with delayed kinetics (data not shown). Scale bars, 50μM. Autofluor., autofluorescence control.

FIG. 9 depicts in accordance with various embodiments of the invention, a time course analysis of iMPC formation. Representative immunofluorescence images for MyHC and Pax7 expression in dox-treated MEFs. Cells were exposed for the indicated lengths of time and cultured in the presence of FR after dox withdrawal. Scale bars, 50μM. Autofluor., autofluorescence control.

FIGS. 10A-10C depict in accordance with various embodiments of the invention, a molecular transcriptome comparison to QSCs and ASCs. Venn-diagram to show overlap of upregulated genes (>2-fold) between quiescent stem cells (QSCs), representing quiescent satellite cells, activated stem cells (ASCs), representing activated satellite cells/myoblasts, and either MEFs+MyoD+FR (FIG. 10A), MEFs+MyoD (FIG. 10B) or MEFs (FIG. 10C). There is overlap of satellite cell-related genes between QSCs, ASCs and the MEFs+MyoD+FR condition. Previous studies demonstrate expression data for QSCs, ASCs and 2 MEF samples used.

FIGS. 11A-11B depict in accordance with various embodiments of the invention, Thy1⁺ fibroblasts are the cell type of origin for iMPCs and testing of the Pax7-CreER lineage tracing system. FIG. 11A) Representative Immunofluorescence images for MyHC and Pax7 expression in sorted Thy1⁻ cells expressing MyoD+FR for 14 days. Scale bars, 100μM. Autofluor., autofluorescence control. FIG. 11B) Representative images of EYFP⁺ iMPCs generated from plated leg muscle of Pax7-CreER×Rosa26-LSL-EYFP mice. Scale bars, 100μM. Autofluor., autofluorescence control.

FIG. 12 depicts in accordance with various embodiments of the invention, FR treatment alone endows myoblast cells with an iMPC phenotype. Quantification of contracting colonies emerging after treatment of the myoblast cell lines C2C12, which expresses endogenous MyoD, with FR.

FIGS. 13A-13B depict in accordance with various embodiments of the invention, surface marker analysis of iMPC cultures. FIG. 13A) Live antibody staining of iMPC cultures for VCAM1, a protein enriched on muscle satellite cells. There is a lack of expression in polynucleated myotubes. FIG. 13B) Live antibody staining in iMPC cultures for CXCR4 and β1-integrin, commonly used to define muscle satellite cells. White arrowheads indicate co-expression of the two surface markers.

FIGS. 14A-14G depict in accordance with various embodiments of the invention, MyoD and small molecules endow fibroblasts with a skeletal muscle progenitor-like state. FIG. 14A) Experimental design assessing if small molecules assist in the reprogramming of MEFs into a proliferative skeletal muscle progenitor-like cell state upon MyoD overexpression. FIG. 14B) Representative bright-field images of cells induced with MyoD in the presence of the indicated small molecules. Basic-FGF (bFGF) was added to all conditions. Three-dimensional, proliferative and contractile colonies were only obtained in the presence of the cyclic AMP agonist Forskolin (F), the TGF-β inhibitor RepSox (R) and Serum Replacement (SR) or ascorbic acid (AA), with or without GSK3β inhibitor (G). This experiment was validated using three different MEF lines; for each replicate 1*10⁵ cells were used per treatment. Scale bars, 500μM. FIG. 14C) Representative Immunofluorescence images of MyHC-positive cells expressing MyoD in the presence of the indicated small molecules in medium containing fetal calf serum (FCS), serum replacement (SR) and basic-FGF (bFGF). Scale bars, 50 μM. FIG. 14D) Ascorbic acid (AA) is critical for the generation of iMPCs and replaces KOSR (SR) supplement. Scale bars, 500μM. FIG. 14E) Representative Immunofluorescence images of MyHC-positive cells expressing MyoD in the absence of SR with and without AA. Scale bars, 50μM. FIG. 14F) Table depicting the quantitative conversion efficiency of MEFs into induced skeletal muscle progenitor-like cells upon MyoD overexpression and exposure to the indicated small molecules. FIG. 14G) Table showing the temporal requirement for MyoD expression to generate skeletal muscle progenitor-like cells in the presence of F/R. Doxycycline and F/R were applied to infected MEFs for the indicated lengths of time. Following dox withdrawal, cells were propagated in the presence or absence of FR and scored for three-dimensional round clusters, cell proliferation and contractility seven days after the last time point (day 12). This experiment was validated using three different MEF lines; for each replicate 1*10⁵ cells were used per time point.

FIGS. 15A-15E depict in accordance with various embodiments of the invention, iMPC cultures grow continuously and express markers for stem, progenitor and mature muscle cells. FIG. 15A) Scheme depicting the differentiation hierarchy within the skeletal muscle system with indication of stage-specific markers. FIG. 15B) Representative Immunofluorescence images for indicated muscle-specific proteins in an iMPC clone. MyHC expressing cells are predominately polynucleated while Pax7 expressing cells are exclusively mononucleated. Scale bars, 50μM. FIG. 15C) Expression of fibroblast-, skeletal muscle-, and cardiac-associated markers by microarray analysis in control MEFs, an established iMPC clone, C2C12 myoblasts and MEFs undergoing conventional transdifferentiation (MEF+MyoD) or reprogramming (MEFs+MyoD+F/R) for 14 days. FIG. 15D) Graphs showing the top upregulated genes by expression microarray in bulk MEF cultures exposed to MyoD+F/R in comparison to MEFs exposed to MyoD alone. Arrows highlight examples of mature muscle markers detected exclusively under reprogramming conditions (MEFs+MyoD+F/R). FIG. 15E) Functional annotation analysis using DAVID for upregulated genes (>2-fold) in MEFs+MyoD+F/R relative to MEFs+MyoD alone. Benjamini-Hochberg (BH) adjusted P values are presented. Top categories are shown together with the number of genes.

FIGS. 16A-16D depict in accordance with various embodiments of the invention, that iMPCs differentiate into myofibers upon transplantation into dystrophic mdx mice. FIG. 16A) Experimental design to assess the engraftment and differentiation potential of iMPCs and control myoblasts into mdx recipient mice. FIG. 16B) Immunofluorescence images for Dystrophin in the indicated samples after transplantation into the tibialis anterior of mdx mice. Muscle sections from the tibialis anterior of a wild type mouse were used as a positive control. FIG. 16C) Immunofluorescence images for Dystrophin and DAPI (punctate staining) showing centrally located nuclei in regenerating Dystrophin-positive myofibers of the indicated samples. FIG. 16D) Quantification of the number of Dystrophin-positive myofibers in tibialis anterior sections from an mdx mouse injected with iMPC clone. The low number of Dystrophin-positive myofibers in non-injected control sections is due to revertant myofibers, which are typically seen in this mouse model.

FIGS. 17A-17G depict in accordance with various embodiments of the invention, iMPC cultures containing satellite-like cells that recapitulate myogenesis in vitro. FIG. 17A) Live antibody staining of iMPC cultures for the satellite cell marker VCAM-1, which is present on mononucleated cells but absent on myotubes. FIG. 17B) Quantitative RT-PCR analysis of MyHC expression in purified VCAM-1⁺Sca1⁻CD31⁻CD45⁻ cells isolated from iMPC cultures and compared to sorted bulk iMPCs immediately after sorting as well as 9 days after sorting and explantation (n=3 biological replicates; error bars s.d.). FIG. 17C) Representative images of the indicated sorted VCAM-1⁺Sca1⁻CD31⁻CD45⁻ or VCAM-1⁻ cells at indicated time points. Only iMPCs form from VCAM-1⁺ cells. Equal numbers of VCAM-1⁺ and VCAM-1 cells were plated for this experiment. FIG. 17D) Representative images of EYFP-positive myotubes from Pax7-CreER×Rosa26-LSL-EYFP MEFs after expression of MyoD and exposure to F/R in the presence of 4-OHT. FIG. 17E) Stable, dox-independent iMPCs develop from Pax7^{+/+} but not Pax7^{-/-} MEFs. Brightfield images show myotubes derived from Pax7^{+/+} and Pax7^{-/-} MEFs upon MyoD overexpression (top) and an iMPC clone derived from Pax7^{+/+} MEFs (bottom left). iMPC-like colonies from Pax7^{-/-} MEFs could not be maintained (bottom right). FIG. 17F) Quantitative RT-PCR analysis for indicated samples. There is upregulation of myogenic genes in Pax7^{+/+} MEFs exposed to MyoD+F/R, but not Pax7^{-/-} MEFs exposed to the same treatment. FIG. 17G) Representative immunofluorescence images show staining for Pax7 in Pax7^{+/+} and Pax7^{-/-} MEFs exposed to MyoD+F/R. Scale bars, 50μM.

FIGS. 18A-18I depict in accordance with various embodiments of the invention, derivation of iMPCs from muscle and MEFs using small molecules alone. FIG. 18A) Experimental design assessing if prolonged small molecule exposure of Pax7-CreER×Rosa26-LSL-EYFP hindlimb muscles (top row) or fibroblasts (bottom row) gives rise to EYFP⁺ iMPCs in the absence of exogenous MyoD expression. FIG. 18B) Representative images of EYFP⁺ iMPCs at passages 0, which were derived from explanted hindlimb muscles of Pax7-CreER×Rosa26-LSL-EYFP mice. Scale bars, 100μM. FIG. 18C) Quantitative RT-PCR analysis for skeletal muscle specific transcripts in sorted EYFP⁺ or EYFP⁻ cells derived from Pax7-CreER;Rosa26-LSL-EYFP MEFs after expression of MyoD and exposure to F/R for 7 days. Untreated MEFs served as negative control (n=3 technical replicates; error bars s.d.). FIG. 18D) Immunofluorescence analysis for Dystrophin expression after injection of iMPCs derived from Pax7-CreER; Rosa26-LSL-EYFP hindlimbs into the tibialis anterior of mdx recipients. Inset to the right shows presence of central nuclei and weak EYFP fluorescence in Dystrophin-positive myofiber using DAPI staining. FIG. 18E) Immunofluorescence images for Dystrophin (red) and Pax7 (green) in the grafts shown in FIG. 18D. Insets indicate nuclear staining for PAX7. FIG. 18F) Flow cytometry analysis of Pax7-CreER×Rosa26-LSL-EYFP MEFs treated with small molecules for 18 days. The PE-Cy7 channel was used to control for autofluorescence. FIG. 18G) Representative image of iMPC clone produced with small molecules. FIG. 18H) Quantitative RT-PCR analysis for skeletal muscle-specific transcripts in iMPC clone generated with small molecules (n=3 technical replicates; error bars s.d.) FIG. 18I) Representative immunofluorescence images show staining for skeletal muscle-specific proteins in iMPC clone derived with small molecules. Scale bars, 100μM.

FIGS. 19A-19D depict in accordance with various embodiments of the invention, the comparison of iMPC transcriptome data with the mouse gene expression database BioGPS. FIG. 19A) Gene expression profile of the housekeeping gene Gapdh across all indicated tissues of the mouse. FIG. 19B) Gene expression profile of the ocular tissue specific gene Crystallin as a positive control across all indicated tissues of the mouse. FIG. 19C) Gene expression profile of the pluripotent stem cell specific gene Nanog as a negative control across all indicated tissues of the mouse. FIG. 19D) Gene expression profile for the most highly expressed genes in iMPCs vs. MEFs across all indicated tissues of the mouse. The indicated genes are expressed specifically in skeletal muscle tissue but not in cardiac tissue.

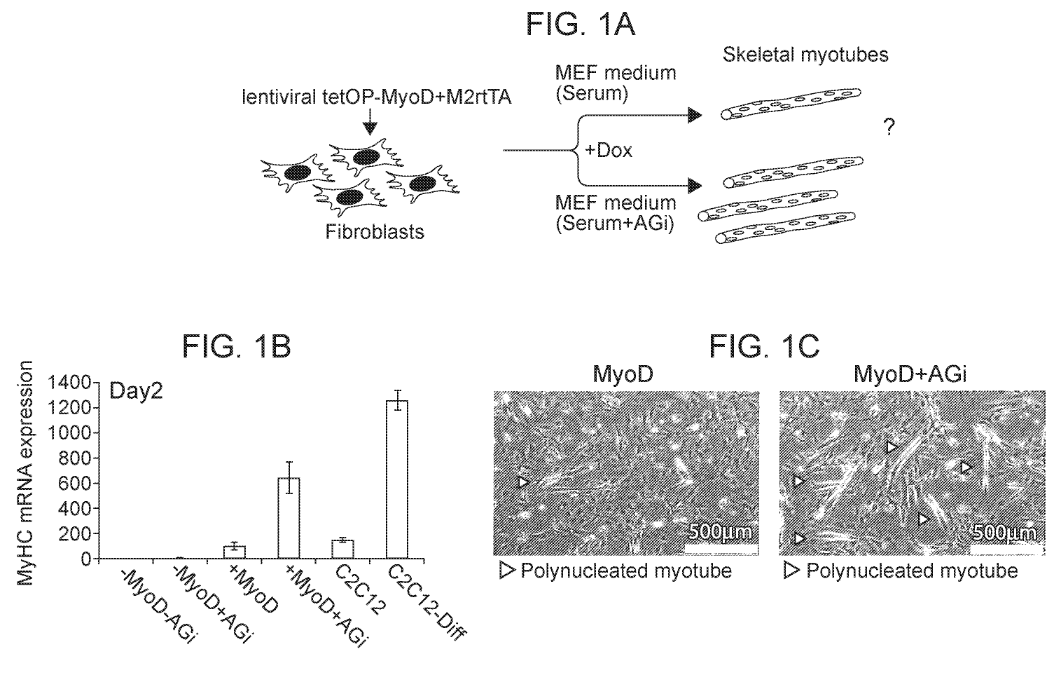

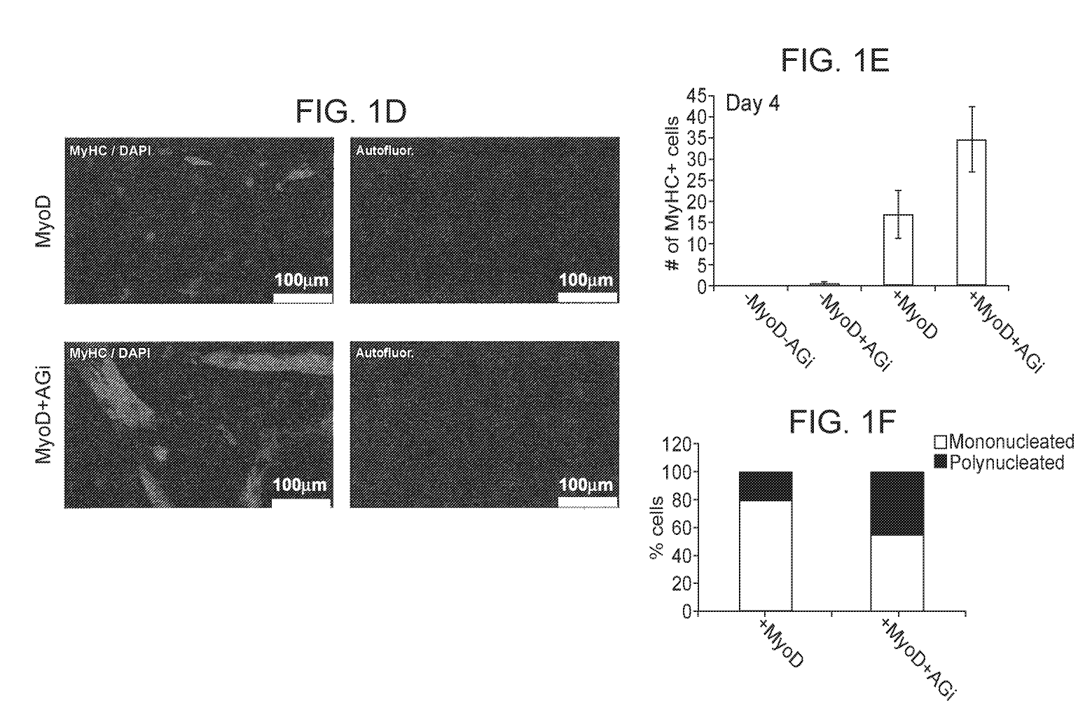

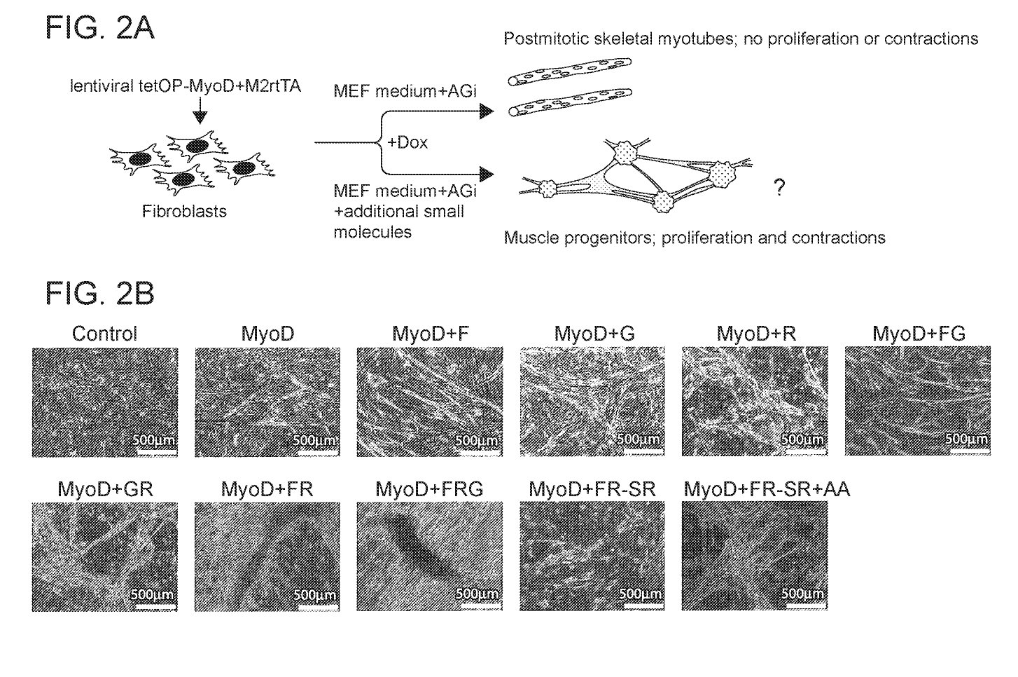

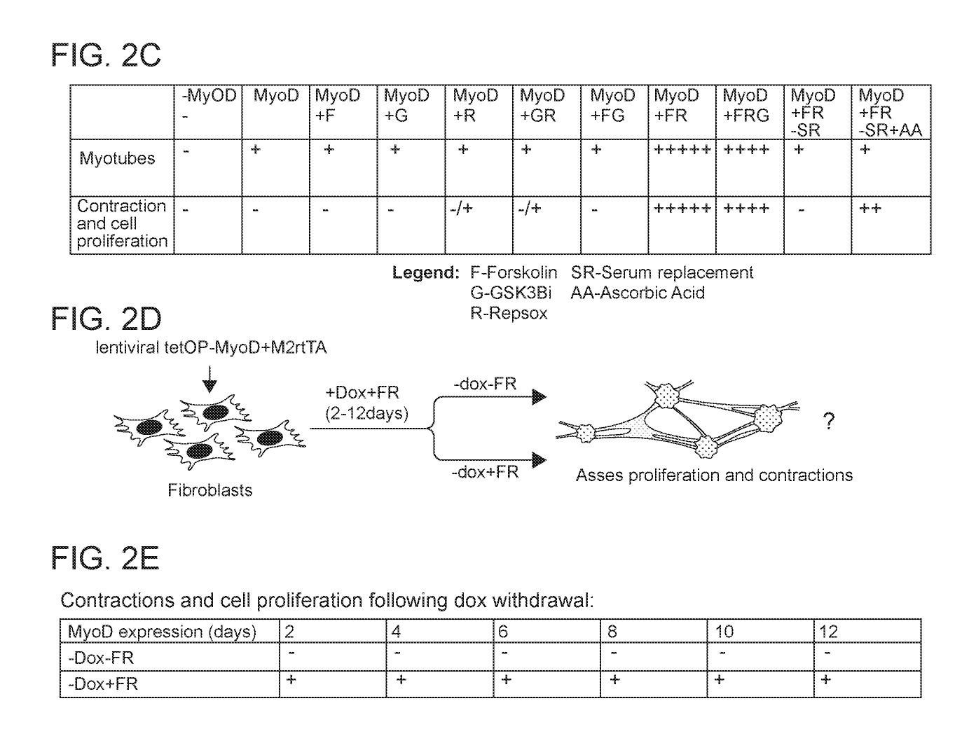

### DETAILED DESCRIPTION

Provided herein are methods, assays and compositions that are derived, in part, from the discovery that muscle progenitor cells can be generated in vitro from somatic cells (e.g., muscle cells, fibroblast cells etc), expanded to numbers useful for therapeutic purposes and can be maintained for long periods of time in culture (e.g., >4 months).

### Definitions

As used herein, the terms “treat,” “treatment,” “treating,” or “amelioration” refer to therapeutic treatments, wherein the object is to reverse, alleviate, ameliorate, inhibit, slow down or stop the progression or severity of a disease or disorder. It will be understood by one of skill in the art that successful treatment does not require complete reversal of the disease or “curing” of the disease. The term “treating” includes reducing or alleviating at least one adverse effect or symptom of a disorder. Treatment is generally “effective” if one or more symptoms or clinical markers are reduced. Alternatively, or in addition, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality. For example, treatment is considered effective if the condition is stabilized. The term “treatment” of a disease also includes providing relief from the symptoms or side-effects of the disease (including palliative treatment).

The term “therapeutically effective amount” refers to an amount of a therapeutic agent and/or a composition comprising a population of cells (e.g., iMPCs or skeletal muscle differentiated therefrom) effective to “treat” a disease or disorder in a subject.

The term “in need thereof” when used in the context of a therapeutic or prophylactic treatment, means having a disease, being diagnosed with a disease, or being in need of preventing a disease, e.g., for one at risk of developing a skeletal muscle disease and/or disorder. Thus, a subject in need thereof can be a subject in need of treating or preventing a disease. In another embodiment, a subject in need thereof can include those presenting with an acute or chronic injury to skeletal muscle from e.g., external trauma, over-use injury, micro- or macro-tears in skeletal muscle fibers or a break-down of muscle tissue (e.g., rhabdomyolysis).

The term “subject” refers to any animal (e.g., a mammal), including, but not limited to humans, non-human primates, rodents, and domestic and game animals, which is to be the recipient of a particular treatment. Primates include chimpanzees, cynomolgous monkeys, spider monkeys, and macaques, e.g., Rhesus. Rodents include mice, rats, woodchucks, ferrets, rabbits and hamsters. Typically, the terms “subject” and “patient” are used interchangeably herein in reference to a human subject. A subject can be male or female. In various embodiments, a subject can be one who has been previously diagnosed with or identified as suffering from or having a condition in need of treatment (e.g., a skeletal muscle disease and/or disorder). In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition.

A subject can be one who has been previously diagnosed with or identified as suffering from a disorder (e.g., muscle-associated disease) and/or injury. A subject can be one who is diagnosed and currently being treated for, or seeking treatment, monitoring, adjustment or modification of an existing therapeutic treatment, or is at a risk of developing a given disorder.

As used herein, “iMPCs” refers to induced myogenic progenitor cells, which are reprogrammed cells that express markers of muscle stem and progenitor cells, can be propagated for at least 3 to 6 months in culture and retain the ability to differentiate and produce contractile myotubes. Examples of markers expressed by iMPCs include, but are not limited to Pax7, Myf5, Cxcr4, Myf6, VCAM1 and Myog. In one embodiment, iMPCs do not have exogenous nucleic acid or a manipulated genetic make-up relative to a somatic cell isolated from an individual.

The term “somatic cells” as used herein refers to cell types in the mammalian body, apart from gametocytes, and undifferentiated stem cells. Examples of somatic cells include, but are not limited to fibroblasts, muscle cells, keratinocytes, melanocytes, and hepatocytes.

“Muscle” as used herein refers to the body tissues which produce force and motion and are formed through myogenesis. Three types of muscle tissue can be produced: skeletal/striated, cardiac and smooth. Muscle fibers generally form from the fusion of myoblasts into multi-nucleated fibers called myotubes. As used herein, the term “muscle cell” refers to a cell of a myogenic lineage and includes satellite cells, myoblasts, myocytes and myotubes.

As used herein, the term “cells derived from a muscle biopsy or muscle explant sample” comprise cells from a skeletal muscle fiber that endogenously express MyoD. In one embodiment, the cells are skeletal muscle cells.

As used herein, “transdifferentiation” refers to a process in which a somatic cell transforms into another somatic cell without undergoing an intermediate pluripotent state or progenitor cell type. As used herein, a transdifferentiation generates a non-proliferative, differentiated cell.

As used herein, the terms “direct reprogramming” and “dedifferentiation”, can be used interchangeably and refer to a process in which a somatic cell is reprogrammed to a proliferative stem/progenitor cell, without passing through a pluripotent state. A directly reprogrammed or dedifferentiated cell, as the term is used herein, is proliferative, can be maintained in culture for at least 4 months, and can be differentiated to a somatic cell of a different phenotype than the original somatic cell when placed under conditions permissive for differentiation. As used herein, a directly reprogrammed or dedifferentiated cell, e.g., an iMPC as described herein, differs from a somatic cell that was induced to a muscle phenotype by expression of MyoD without a cocktail as described herein in that the resulting cells have a muscle progenitor cell phenotype and are proliferative, rather than being fully differentiated and lacking proliferative activity or capacity.

As used herein, “transient expression” refers to the temporary expression of agents administered to aid in a cellular phenotypic change, such as but not limited to, transcription factors and growth factors. Transient expression can be achieved in a number of ways, including, but not limited to expression from an inducible expression construct.

The terms “increased,” or “increase” are used herein to generally mean an increase by a statically significant amount; for the avoidance of doubt, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. In other embodiments, the term “increased” means an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. For example, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages means ±1% of the value being referred to. For example, about 100 means from 99 to 101.

Repair of skeletal muscle in response to injury comprises the activation of satellite cells within the skeletal muscle fiber, which then fuse with existing skeletal muscle cells or with other satellite cells to repair and/or regenerate damaged muscle fibers. While satellite cells and myoblasts can be transiently cultured and modestly expanded using growth factors or small molecules, current protocols do not allow for the long-term maintenance of primary, non-transformed stem/progenitor cells with myogenic potential ex vivo.

The different stages of adult myogenesis are distinguished by the expression of distinct transcription factors or surface markers. For example, quiescent satellite cells express the transcription factor Pax7 and the surface marker VCAM1 but lack expression of the myogenic determination protein 1 (MyoD). By contrast, activated satellite cells (i.e., myoblasts) co-express Pax7 and MyoD, whereas differentiating myoblasts and myotubes upregulate other myogenic factors such as myogenic regulatory factor 4 (MRF4 or Myf6) and Myogenin (MyoG) in addition to MyoD. Pax7 expression serves as a useful marker for quiescent and activated satellite cells and is often used to genetically mark or purify these immature cell populations using fluorescent reporters or lineage tracing alleles. Moreover, Pax7 expression is functionally required for the specification and maintenance of the adult satellite cell pool as well as for muscle repair.

Given previous studies on transcriptional regulators important for the different stages of myogenesis, without being bound by any particular theory, the inventors reasoned that it could be feasible to induce muscle stem or progenitor-like cells from heterologous somatic cell types using cellular reprogramming. Indeed, the generation of myotubes from fibroblasts upon ectopic expression of the transcription factor MyoD represents the first example of “direct lineage conversion” or “transdifferentiation” in a mammalian system. These studies provided the framework for subsequent attempts to convert one mature cell type into another (e.g., murine embryonic fibroblasts (MEFs) to neurons, MEFs to cardiomyocytes, B cells to macrophages). While these approaches have been important to dissect the mechanisms by which transcription factors control cell fate, they are limited in that post-mitotic, non-expandable cells are typically generated. This is particularly problematic for potential clinical settings where millions to billions of mature cells may be required to achieve a therapeutic benefit in patients. Although the transplantation of fibroblasts carrying a MyoD-inducible transgene has been proposed as a source of replacement muscle cells in vivo, this approach also generates post-mitotic cells, involves genetic manipulation and requires treatment of mice with tamoxifen. Induced pluripotent stem cells (iPSCs) may provide an alternative solution as they can be expanded indefinitely and differentiated repeatedly into myogenic cells using recently developed protocols. However, myogenic stem/progenitor cells derived from iPSCs are difficult to maintain in culture and current technology does not allow permanent capture of these cell populations in vitro. Moreover, residual pluripotent cells may form teratomas upon transplantation, complicating their therapeutic utility.

As described herein, the inventors have demonstrated that ectopic expression of the myogenic transcription factor MyoD, combined with exposure to three small molecules, readily reprograms somatic cells, such as fibroblasts (e.g., mouse fibroblasts) into “induced myogenic progenitors” (iMPCs) that can be propagated for at least 3 to 6 months, while retaining the ability to produce contractile myotubes when placed under conditions that permit or promote differentiation. Immature iMPCs express markers of muscle stem and progenitor cells, including Pax7 and Myf5, and can differentiate into Dystrophin expressing myofibers upon transplantation into a mouse model of Duchenne's Muscular Dystrophy. The inventors also show that iMPCs and derivative myotubes originate from Pax7+ stem-like cells and do not pass through a transient Oct4+ pluripotent state. The inventors further demonstrate that iMPC maintenance requires the master regulator Pax7, underscoring functional similarities with satellite cells in vivo. Lastly, evidence that functional iMPCs can be generated from explanted muscle or skin tissue following small molecule exposure alone is provided; that is, while it increases efficiency, MyoD expression is not required for the production of iMPCs from somatic cells. These findings reveal a novel and facile approach to derive expandable myogenic stem/progenitor cells with characteristics of satellite cells from different somatic tissues.

The present invention is based, at least in part, on these findings. Embodiments address the need in the art for methods of generating a proliferative or self-renewing population of muscle progenitor cells or induced muscle progenitor cells (iMPCs). Embodiments further provide for methods of promoting muscle regeneration and/or repair, and methods of treating a muscle disease or disorder.

Method of Generating iMPCs

Various embodiments of the present invention provide for a method of generating induced muscle progenitor cells (iMPCs), the method comprising: contacting a population of somatic cells obtained from a subject with a cyclic AMP agonist and a TGF-β inhibitor for a time and under conditions that induce dedifferentiation of the somatic cells to a population of cells comprising iMPCs.

In one embodiment, the somatic cells are fibroblasts or skeletal muscle cells. Additional somatic cell types for use with the compositions and methods described herein include: a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin, adipose or muscle biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, lung, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

In embodiments where the somatic cells are derived from non-muscle cells that do not express a myogenic factor, such as MyoD, endogenously the method can further comprise expressing an exogenous myogenic factor in the somatic cells. In some embodiments, the somatic cells are muscle biopsy or muscle-derived explants and the iMPCs are muscle-induced iMPCs (M-iMPCs).

In some embodiments, the exogenous myogenic factor is MyoD. In other embodiments, the exogenous MyoD is expressed transiently. In yet other embodiments, the exogenous MyoD is expressed for a minimum of 2 days. Alternatively, cells derived from a muscle biopsy or muscle cell explant that endogenously express MyoD do not require the exogenous expression of MyoD in order to be successfully dedifferentiated into iMPCs, as that term is used herein. In one embodiment, exogenous MyoD is not expressed for more than 4 days.

Essentially any cyclic AMP agonist and/or TGF-β inhibitor can be used in the methods described herein. In one embodiment, the cyclic AMP agonist is forskolin. In some embodiments, the TGF-β inhibitor is RepSox, SB-431542 or ALK5 Inhibitor II. In one embodiment, the TGF-β inhibitor is RepSox. Additional non-limiting examples of small molecule inhibitors of TGF-β receptors include 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), and SB-505124 (see e.g., Dacosta Byfield, S., et al., 2004; Molecular Pharmacology 65:744-52), among others. Additional TGF-β receptor antagonists are known in the art.

In various embodiments, the method further comprises culturing the somatic cells and/or population of cells comprising iMPCs with ascorbic acid.

In various embodiments, the method further comprises a step of isolating an iMPC and plating it as a clonal culture. That is, a population of somatic cells is treated to induce dedifferentiation into iMPCs, individual iMPCs are detected using morphology or cell surface marker expression, a desired individual iMPC is then removed from the original culture and serially replated to produce a substantially homogeneous population of iMPCs comprising substantially similar structural and/or functional properties.

In various other embodiments, the iMPCs are proliferative, self-renewing and capable of forming skeletal muscle myotubes. In some embodiments, the iMPCs can be maintained in culture (e.g., without substantial loss of their self-renewal or ability to differentiate into skeletal myotubes) for at least 4 months (e.g., at least 5, at least 6, at least 7, at least 8, at least 9, at least 10, at least 11, at least 12 months or more). In other embodiments the iMPCs can be maintained in culture without loss of their self-renewal and ability to differentiate into myotubes for at least 1 year at least 18 months, at least 24 months or more. In one embodiment, the iMPCs can be maintained in culture for greater than 6 months.

In various embodiments, the population of cells derived from dedifferentiation of somatic cells as described herein is a heterogeneous culture of cells. In some embodiments, the population of cells further comprises differentiated skeletal muscle cells. In other embodiments, the dedifferentiation of the somatic cells to iMPCs does not go through a transient pluripotent state. In some embodiments, the iMPC cell or population comprising such iMPCs expresses one or more of the following markers: Pax7, Myf5, Cxcr4, Myf6, VCAM1, Myog and MyHC. In various other embodiments, the iMPCs do not detectably express fibroblast markers. In some embodiments, the fibroblast markers are Col5a1, Thy1, and Fbln5. In other embodiments, the iMPCs are mononucleated.

Various embodiments of the present invention also provide for an in vitro heterogeneous population of skeletal muscle cells comprising induced muscle progenitor cells (iMPCs). In various embodiments, the heterogeneous population can be maintained in culture without loss of phenotype for at least 6 months. In various other embodiments, the in vitro heterogeneous population further comprises medium comprising ascorbic acid, GSK3 inhibitor and FGF.

Described herein are methods to derive and establish iMPCs from somatic cells with the beneficial characteristics of: i) maintaining the cells in culture, ii) preserving the cells' myogenic potential and iii) the capability of passaging them in culture for a long period of time.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Various embodiments of the present invention provide for the generation of induced muscle progenitor cells (iMPCs) using a medium comprising a cyclic AMP agonist and a TGF-β inhibitor. In various embodiments, a molecule that increases cAMP levels is administered as the cyclic AMP agonist. In various embodiments, the cyclic AMP agonist is forskolin. In other embodiments, forskolin can be used at a concentration of 1μM to 10 μM, inclusive. In various embodiments, the concentration of forskolin is between 1μM-9μM, 1μM-8μM, 1μM-7μM, 1μM-6μM, 1μM-5μM, 1μM-4μM, 1μM-3μM, 1μM-2μM, 2μM-3μM, 3μM-6μM, 6μM-8μM, 2μM-10μM, 3μM-10μM, 4μM-10μM, 5μM-10μM, 6μM-10μM, 7 μM-10μM, 8μM-10μM, or 9μM-10μM. In one embodiment, the concentration of forskolin is 5 μM. Other cAMP agonists can also be used (e.g., including, but not limited to (3₂-adrenergic agonists such as salbutamol, salmeterol and propranolol; PGI₂ analogs such as treprostinil; 8-(6-Aminohexyl)aminoadenosine 3′:5′-cyclic monophosphate) and N-Acetyl-5-hydroxytryptamine. Such agents can be used at a concentration that provides cAMP levels within the ranges provided by treatment with 1 μM to 10 μM forskolin. Alternatively, or in addition, one of ordinary skill in the art can readily determine a concentration of a cAMP agonist other than forskolin that provides activity in dedifferentiation similar to that of forskolin by testing the cAMP agonist over a range of concentrations while keeping other members of the cocktail constant and monitoring iMPC emergence as described herein.

Examples of TGF-β inhibitors include, but are not limited to RepSox, SB431542 or an ALK5 Inhibitor II (EMD616452). In various embodiments, the TGF-β inhibitor is RepSox (2-3 [(6-methyl-2-pyridinyl)-1H-pyrazol-4-yl]-1,5-napthyridine). In other embodiments, the concentration of TGF-β inhibitor is an amount that gives TGF-β inhibition in the range provided by RepSox (e.g., 1μM to 10 μM, inclusive). In other embodiments, the concentration of TGF-β inhibitor is an amount that gives TGF-β inhibition of at least 25%, at least 50%, at least 75%, at least 80%, at least 90%, at least 95%, at least 98%, at least 99% as compared to the TGF-β inhibition by RepSox (e.g., at a concentration of 1μM to 10 μM, inclusive). Alternatively, or in addition, one of ordinary skill in the art can readily determine a concentration of a TGF-β inhibitor other than RepSox that provides activity in dedifferentiation similar to that of RepSox by testing the TGF-β inhibitor over a range of concentrations while keeping other members of the cocktail constant and monitoring iMPC emergence as described herein. In various embodiments, the concentration of TGF-β inhibitor is between 1μM-9μM, 1 μM-8μM, 1 μM-7μM, 1μM-6μM, 1μM-5μM, 1μM-4μM, 1μM-3μM, 1μM-2μM, 2μM-3μM, 3μM-6μM, 6μM-8μM, 2 μM-10μM, 3μM-10μM, 4μM-10μM, 5 μM-10μM, 6μM-10μM, 7 μM-10μM, 8 μM-10 μM, or 9μM-10μM. In one embodiment, the concentration of TGF-β inhibitor is 5μM.

In various embodiments, the medium further comprises ascorbic acid. In some embodiments, the concentration of ascorbic acid is between 20μg/ml and 100μg/ml. In various embodiments, the concentration of ascorbic acid is between 20μg/ml-90μg/ml, 20μg/ml-80μg/ml, 20μg/ml-75μg/ml, 20μg/ml-50μg/ml, 20μg/ml-25 μg/ml, 20μg/ml-40 μg/ml, 40μg/ml-60μg/ml, 40μg/ml-100 μg/ml, 60 μg/ml-80 μg/ml, 60 μg/ml-100μg/ml, or 80μg/ml-100μg/ml. In one embodiment, the concentration of ascorbic acid is 50μg/ml. In various embodiments, the medium comprises In various embodiments, ascorbic acid is useful in the reprogramming of the somatic cells to iMPCs. In other embodiments, ascorbic acid is useful in the propagation and/or maintenance of the iMPCs.

In various embodiments, the medium further comprises a GSK3β inhibitor. While not required, the addition of a GSK3β inhibitor to the cell culture boosts formation of iMPCs. Examples of GSK3β inhibitors include, but are not limited to ATP-Competitive GSK-33 Inhibitors, such as Pyrazolopyrimidines, Benzimidazoles, Pyridinones, Pyrimidines, Indolylmaleimide, Imidazopyridines, Oxadiazoles, Pyrazines; and Non-ATP-Compestitive GSK-303 Inhibitors, such as 5-Imino-1,2,4-Thiadiazoles (ITDZs). Further examples of a GSK303 inhibitor include, but are not limited to CHIR99021, 6-bromoindirubin-3′-oxime (Bio), and IM-12. In various embodiments, the GSK3β inhibitor is CHIR99021. In various other embodiments, the concentration of the GSK3β inhibitor is between 1μM and 20μM or is in an amount sufficient to inhibit GSK3β to within 25% of the inhibition provided by CHIR99021 at a concentration of 1 μM to 20μM, inclusive. Alternatively, or in addition, one of ordinary skill in the art can readily determine a concentration of a GSK3β inhibitor other than CHIR99021 that provides activity in dedifferentiation similar to that of CHIR99021 by testing the GSK3β inhibitor over a range of concentrations while keeping other members of the cocktail constant and monitoring iMPC emergence as described herein. In some embodiments, the concentration of the GSK3β inhibitor is between 1μM-4μM, 4μM-8μM, 8μM-12μM, 12μM-16μM, or 16μM-20μM. In various embodiments, the concentration of the GSK3β inhibitor is 3μM. In various other embodiments, the concentration of the GSK3β inhibitor is 10μM. In various other embodiments, the medium comprises molecules that are activated by GSK3β inhibition. In various embodiments, Wnt growth factors are the molecules that are activated by GSK3β inhibition, and it is contemplated that other Wnt activators as known to those of ordinary skill in the art could also provide a benefit in boosting iMPC production similar to that provided by CHIR99021.

In various embodiments, the medium further comprises a fibroblast growth factor (FGF), such as basic FGF (bFGF). In various other embodiments, the FGF is basic FGF (bFGF) or acidic FGF. In yet other embodiments, the FGF is bFGF. In various embodiments, the concentration of bFGF is between 1 ng/ml-20 ng/ml, inclusive. In various other embodiments, the concentration of bFGF is between 1 ng/ml-5 ng/ml, 5 ng/ml-10 ng/ml, 10 ng/ml-15 ng/ml or 15 ng/ml-20 ng/ml. In some embodiments, the concentration of bFGF is 10 ng/ml.

In various embodiments, MyoD is added via cells genetically modified with an inducible vector system or directly added to the culture for at least one day (e.g., 1, 2, 3 days or more). It is important to note that while MyoD has long been known to induce a myogenic phenotype when ectopically expressed in different stem cells or even somatic cells of another lineage, the myogenic cells that result are not proliferative—this is in sharp contrast to the cells generated with the cocktail described herein, the efficiencies of muscle progenitor generation is enhanced by the transient expression of MyoD. In various embodiments, MyoD exposure in combination with the small molecule cocktail described herein results in iMPCs in about 1 week. In various other embodiments, only the small molecules were added to the culture—i.e., no genetic manipulation to express MyoD was performed. In some embodiments, only small molecule exposure resulted in iMPCs in about 3 weeks. In other embodiments, the small molecules are forskolin, RepSox, CHIR99021, ascorbic acid, FGF or a combination thereof. In yet other embodiments, only the small molecules are used for the expansion of the iMPCs for a prolonged period of time (e.g., weeks to months in culture).

The composition of the medium described herein, comprises a combination of small molecules and transcription factors. While some of the molecules described herein can be associated with muscle differentiation, the combination of the medium described herein results in a proliferating progenitor cell. Compared to previous studies that used some of these molecules or cytokines, the inventors demonstrated for the first time that (i) a change of cell fate (fibroblast to muscle) as well as a gain in differentiation potential (differentiated cell to progenitor cell) is achieved and (ii) indefinite proliferation of muscle progenitors is attained.

In various embodiments, the iMPC cells generated can be genetically modified to introduce one or more polynucleotides encoding one or more proteins or chimeric proteins that label the cells. Thus, in certain embodiments, the iMPCs are genetically modified to encompass a label for identification. In various embodiments, the labeled cells can be used to monitor the progression of treatment. Examples of labels are known in the art and include, but are not limited to, green fluorescent protein (GFP), yellow fluorescent protein (YFP), blue fluorescent protein (BFP), and/or cyan fluorescent protein (CFP). If so desired, the iMPCs can also be genetically modified to express a desired transgene or transgene expression system, e.g., to provide a function other than, or in addition to, labeling the cells.

The iMPCs can be transfected using any of numerous RNA or DNA expression vectors known to those of ordinary skill in the art. Genetic modification can comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York. N.Y. The administered cells can also be transduced using viral transduction methodologies such as, but not limited to retroviral or lentiviral transduction technologies, which are known in the art.

### Scaffold Compositions

Biocompatible synthetic, natural, as well as semi-synthetic polymers, can be used for synthesizing polymeric particles that can be used as a scaffold material for e.g., seeding iMPCs for therapeutic treatment. In general, for the practice of the methods described herein, it is preferable that a scaffold biodegrades such that the iMPCs can be isolated from the polymer prior to implantation or such that the scaffold degrades over time in a subject and does not require removal. Thus, in one embodiment, the scaffold provides a temporary structure for growth and/or delivery of iMPCs to a subject in need thereof. In some embodiments, the scaffold permits human muscle progenitors to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, fibrin, silk, synthetic polyamino acids and prolamines; polysaccharides such as alginate, heparin; and other naturally occurring biodegradable polymers of sugar units. Alternately, combinations of the aforementioned polymers can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature.

PGA is a homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in the matrix should meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy.

Scaffolds can be of any desired shape and can comprise a wide range of geometries that are useful for the methods described herein. A non-limiting list of shapes includes, for example, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, agar, agarose, gelatin, gum arabic, collagens types I, II, III, IV, and V, fibronectin, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen.

In some embodiments it can be desirable to add bioactive molecules to the scaffold. A variety of bioactive molecules can be delivered using the matrices described herein. These are referred to generically herein as “factors” or “bioactive factors”.

In one embodiment, the bioactive factors include growth factors. Examples of growth factors include platelet derived growth factor (PDGF), transforming growth factor alpha or beta (TGFβ), bone morphogenic protein 4 (BMP4), fibroblastic growth factor 7 (FGF7), fibroblast growth factor 10 (FGF10), epidermal growth factor (EGF/TGFα), vascular endothelium growth factor (VEGF), some of which are also angiogenic factors.

These factors are known to those skilled in the art and are available commercially or described in the literature. Bioactive molecules can be incorporated into the matrix and released over time by diffusion and/or degradation of the matrix, or they can be suspended with the cell suspension.

### Methods of Treatment

Various embodiments of the present invention also provide for a method for promoting muscle regeneration and/or repair, the method comprising: administering a therapeutically effective amount of iMPCs to a subject in need thereof. In various embodiments, the iMPCs are prepared according to the methods described herein. In various other embodiments, the iMPCs are autologous to the subject. Alternatively, the cells can be allogenic to the recipient. In yet other embodiments, the therapeutically effective amount comprises at least 1×10⁵ cells. In other embodiments, the therapeutically effective amount comprises at least 1×10⁶ cells. For use in the various aspects described herein, a therapeutically effective amount of iMPCs comprises at least 10² iMPCs, at least 5×10², at least 10³, at least 5×10³, at least 10⁴, at least 5×10⁴, at least 10⁵, at least 2×10⁵, at least 3×10⁵, at least 4×10⁵, at least 5×10⁵, at least 6×10⁵, at least 7×10⁵, at least 8×10⁵, at least 9×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶ iMPCs or more.

Various embodiments of the present invention also provide for a method for treating a muscle disease or disorder, the method comprising: administering a therapeutically effective amount of iMPCs to a subject in need thereof. In various embodiments, the iMPCs are prepared according to the methods described herein. In various embodiments, the iMPCs are autologous to the subject. Alternatively, the cells can be allogenic to the recipient. In various other embodiments, the therapeutically effective amount comprises at least 1×10⁵ cells. In some embodiments, the therapeutically effective amount comprises at least 1×10⁶ cells. In yet other embodiments, the muscle disease or disorder is characterized by a gene mutation and/or deficiency.

In various embodiments, the disease is a muscle-associated disease. In various other embodiments, the disease is characterized by or involves muscle degeneration or atrophy. Examples of muscle-associated diseases or disorders include, but are not limited to, muscular dystrophy, such as, Duchenne's muscular dystrophy, Becker muscular dystrophy, facioscapulohumeral muscular dystrophy, and myotonic dystrophy, congenital muscular dystrophy, distal muscular dystrophy, Emery-Dreifuss muscular dystrophy, oculopharyngeal muscular dystrophy, limb girdle muscular dystrophy; congenital myopathies, such as, central core, mryotubular, nemaline, Ullrich/ethlem, RyR11 and metabolic muscle diseases, such as, mitochondrial myopathy, Pompe disease, McArdle's disease, and carnitine palmitoyl transferase deficiency. In various embodiments, the disease is a muscle wasting disease. In other embodiments, the disease is a muscle aging disease. In yet other embodiments, the disease results in muscle loss.

Also contemplated herein is the treatment of acute or chronic muscle injury resulting from e.g., break-down of skeletal muscle (e.g., rhabdomyolysis), traumatic injury (e.g., auto accidents or any other trauma that could slice, cut or otherwise damage a muscle fiber), or over-use injuries, etc.

### Dosage and Administration

The generation of iMPCs described herein is useful, for example, in a variety of applications including, but not limited to, promoting muscle regeneration and/or repair, and/or treating a muscle disease or disorder. The methods of use can be in vitro, ex vivo, or in vivo methods. In certain embodiments, the iMPCs are genetically modified to encompass a label for identification. Examples of labels include, but are not limited to, GFP, YFP, BFP, and/or CFP.

In various embodiments, the iMPCs generated according to the invention can be formulated for delivery via any route of administration. “Route of administration” may refer to any administration pathway known in the art, including but not limited to parenteral.

“Parenteral” refers to a route of administration that is generally associated with injection, including infusion, intraarterial, intracapsular, intracardiac, intradermal, intramuscular, intraperitoneal, intraspinal, intrasternal, intrathecal, intrauterine, intravenous, subarachnoid, subcapsular or subcutaneous. Via the parenteral route, the iMPC cell composition can be combined with solutions or suspensions for infusion or for injection.

In various embodiments, the iMPC cell composition can be administered in a matrix, e.g., a collagen matrix or other matrix. The matrix comprises a decellularized scaffold, e.g., produced by decellularizing a donor tissue Methods for obtaining decellularized tissue matrices using physical, chemical, and enzymatic means are known in the art, see, e.g., Liao et al, Biomaterials 29(8): 1065-74 (2008); Gilbert et al, Biomaterials 27(9):3675-83 (2006); Teebken et al, Eur. J. Vase. Endovasc. Surg. 19:381-86 (2000). See also U.S. Pat. Publication Nos. 20130084266, 2009/0142836; 2005/0256588; 2007/0244568; and 2003/0087428. In various embodiments, the iMPC cell composition can be administered intramuscularly by injection or by gradual infusion over time. Given an appropriate formulation for a given route, for example, the iMPC cell composition useful in the methods described herein can be administered, e.g., artificially prepared tissues produced by expansion and differentiation of iMPCs in culture, alone or in conjunction with other cells and/or a scaffold comprising extracellular materials that can be implanted at a desired site intradermally, intramuscularly, or subcutaneously, and can be delivered by peristaltic means, if desired, or by other means known by those skilled in the art.

The iMPC cell composition according to the invention can also contain a pharmaceutically acceptable carrier. “Pharmaceutically acceptable carrier” as used herein refers to a pharmaceutically acceptable material, composition, or vehicle that assists in establishing or maintaining the iMPC cell composition in a form for administration. For example, the carrier may be a liquid filler, diluent, excipient, or solvent, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, the present invention provides pharmaceutical compositions including a pharmaceutically acceptable excipient along with a therapeutically effective amount of the iMPC cell composition. “Pharmaceutically acceptable excipient” means an excipient that is useful in preparing the iMPC cell composition that is generally safe, non-toxic, and desirable, and includes excipients that are acceptable for veterinary use as well as for human pharmaceutical use. The active ingredient, e.g., cells, can be mixed with excipients which are pharmaceutically acceptable and compatible with the active ingredient and in amounts suitable for use in the therapeutic methods described herein. To the extent compatible with the cells, a cell composition as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Physiologically tolerable carriers are well known in the art. The amount of an active agent used in the invention that will be effective will depend on the nature of the disorder or condition, and can be determined by one of skill in the art with standard clinical techniques.

The iMPC cell composition as described herein can be administered either alone, or as a cell composition in combination with diluents and/or with other components such as transcription factors, cytokines or other cell populations. The iMPC cell composition can comprise a combination of one or more pharmaceutically or physiologically acceptable carriers, diluents or excipients, discussed above.

The iMPC cell composition according to the invention can be delivered in an “effective amount.” The precise therapeutically effective amount is that amount of the composition that will yield the most effective results in terms of efficacy of promoting muscle regeneration and/or repair, and/or treating a muscle disease or disorder in a subject. This amount will vary depending upon a variety of factors, including but not limited to the characteristics of the iMPC cell composition (including stage of differentiation and activity), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the art will be able to determine a therapeutically effective amount through routine experimentation, for instance, by monitoring a subject's response to administration of the cell composition and adjusting the dosage or administration regimen, accordingly.

Typical dosages of an effective iMPC cell composition can be as indicated to the skilled artisan by the in vitro responses or responses in animal models or cell culture. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells, cell lines or histocultured tissue sample, such as biological samples obtained, or the responses observed in the appropriate animal models. As discussed above, in some embodiments, the iMPC cell composition administered can be between 1×10⁴ to 1×10¹¹ cells. In some embodiments, the iMPC cell composition administered is between 1×10⁵ to 1×10⁶ cells. In some embodiments, the iMPC cell composition administered is at least 1×10⁵ cells. In some embodiments, the iMPC cell composition administered is at least 1×10⁶ cells. In some other embodiments, the number of cells administered can be greater than 1×10¹⁰ cells.

For the treatment of muscle-associated diseases or disorders, the appropriate dosage of the iMPC cell compositions of the present invention depends on the type of disease to be treated, the severity and course of the disease, the responsiveness of the disease, whether the cell composition is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates.

The cell compositions can be administered one time or over a series of administrations. The cell compositions of the present invention can be administered in multiple, sequential dosages as determined by a clinician.

As used herein, the term “administering,” refers to the placement of a therapeutic composition comprising iMPCs as disclosed herein into a subject by a method or route that results in at least partial delivery of the cell composition at a desired site.

The efficacy of compositions as described herein in, e.g., the treatment of a condition described herein can be determined by the skilled clinician. However, a treatment is considered an “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., increased muscle regeneration or increase in gene expression (e.g., dystrophin). Efficacy can also be measured by a failure of an individual to worsen as assessed by hospitalization, or need for medical interventions (i.e., progression of the disease is halted or slowed). Methods of measuring these indicators are known to those of skill in the art and/or are described herein.

Efficacy can be assessed in animal models of a condition described herein, for example, a mouse model of muscular dystrophy, or an appropriate animal model for muscle degeneration, as the case may be. When using an experimental animal model, efficacy of treatment is evidenced when a statistically significant change in a marker is observed.

### Method of Drug Screening and Disease Modeling

Various embodiments of the present invention provide for a method of screening for a drug useful in the treatment of a disease comprising obtaining a somatic cell sample from a subject with the disease; generating iMPCs by the methods disclosed herein; contacting the iMPCs generated with a drug, and; determining the effect of the drug on the iMPC cells.

In some embodiments, the iMPCs as generated by the methods described herein can be used in methods, assays, systems and kits to develop specific in vitro assays. Such assays for drug screening and toxicology studies have an advantage over existing assays because they are of human origin, and do not require immortalization of cell lines, nor do they require tissue from cadavers, which poorly reflect the physiology of normal human cells. For example, the methods, assays, systems, and kits described herein can be used to identify and/or test agents that can repair and/or regeneration of skeletal muscle cells, myotubes and/or myofibers. In addition to, or alternatively, the methods, assays, systems, and kits can be used to identify and/or test for agents useful in treating a muscle disease or disorder, or for treating skeletal muscle injury.

Accordingly, provided herein are methods for screening a test compound for biological activity, the method comprising (a) contacting an iMPC or heterogeneous population comprising iMPCs as described herein, or its progeny, with a test compound and (b) determining any effect of the compound on the cell. In one embodiment, the screening method further comprises generating a iMPC or heterogeneous population comprising iMPCs as disclosed herein. The effect on the cell can be one that is observable directly or indirectly by use of reporter molecules.

As used herein, the term “biological activity” or “bioactivity” refers to the ability of a test compound to affect a biological sample. Biological activity can include, without limitation, elicitation of a stimulatory, inhibitory, regulatory, toxic or lethal response in a biological assay. For example, a biological activity can refer to the ability of a compound to modulate the effect of an enzyme, block a receptor, stimulate a receptor, modulate the expression level of one or more genes, modulate cell proliferation, modulate cell division, modulate cell metabolism, modulate differentiation, modulate cell morphology, or a combination thereof. In some instances, a biological activity can refer to the ability of a test compound to produce a toxic effect in a biological sample.

As used herein, the term “test compound” or “candidate agent” refers to an agent or collection of agents (e.g., compounds) that are to be screened for their ability to have an effect on the cell. Test compounds can include a wide variety of different compounds, including chemical compounds, mixtures of chemical compounds, e.g., polysaccharides, small organic or inorganic molecules (e.g. molecules having a molecular weight less than 2000 Daltons, less than 1000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds can be provided free in solution, or can be attached to a carrier, or a solid support, e.g., beads. A number of suitable solid supports can be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

A number of small molecule libraries are known in the art and commercially available. These small molecule libraries can be screened using the screening methods described herein. A chemical library or compound library is a collection of stored chemicals that can be used in conjunction with the methods described herein to screen candidate agents for a particular effect. A chemical library comprises information regarding the chemical structure, purity, quantity, and physiochemical characteristics of each compound. Compound libraries can be obtained commercially, for example, from Enzo Life Sciences™, Aurora Fine Chemicals™, Exclusive Chemistry Ltd.™, ChemDiv, ChemBridge™, TimTec Inc.™, AsisChem™, and Princeton Biomolecular Research™, among others.

Without limitation, the compounds can be tested at any concentration that can exert an effect on the cells relative to a control over an appropriate time period. In some embodiments, compounds are tested at concentrations in the range of about 0.01 nM to about 100 mM, about 0.1 nM to about 500μM, about 0.1 μM to about 20μM, about 0.1 μM to about 10μM, or about 0.1 μM to about 5μM.

The compound screening assay can be used in a high through-put screen. High through-put screening is a process in which libraries of compounds are tested for a given activity. High through-put screening seeks to screen large numbers of compounds rapidly and in parallel. For example, using microtiter plates and automated assay equipment, a laboratory can perform as many as 100,000 assays per day in parallel.

The compound screening assays described herein can involve more than one measurement of the cell or reporter function (e.g., measurement of more than one parameter and/or measurement of one or more parameters at multiple points over the course of the assay). Multiple measurements can allow for following the biological activity over incubation time with the test compound. In one embodiment, the reporter function is measured at a plurality of times to allow monitoring of the effects of the test compound at different incubation times.

The screening assay can be followed by a subsequent assay to further identify whether the identified test compound has properties desirable for the intended use. For example, the screening assay can be followed by a second assay selected from the group consisting of measurement of any of: bioavailability, toxicity, or pharmacokinetics, but is not limited to these methods.

In some embodiments, the subjects somatic cells are isolated, iMPCs generated and the cells assessed, for example, for use in generating a personalized treatment regimen. In other embodiments, the iMPCs generated from the subject undergo drug screening by the method described herein. In other embodiments, the somatic cells from the subject undergo small molecule screening to determine the optimal small molecule combination and concentration to obtain a personalized iMPC.

### Kits

The present invention is also directed to a kit to generate or maintain iMPCs from somatic cells and/or to treat a subject in need of iMPCs, and/or to differentiate iMPCs to skeletal muscle. In one embodiment, the kit is an assemblage of materials or components useful to perform the dedifferentiation of a somatic cell to an iMPC as described herein. In another embodiment, the kit contains a composition including a cyclic AMP agonist and a TGF-β inhibitor, and optionally an exogenous myogenic factor or construct for expression thereof, and/or ascorbic acid, as described herein. In one embodiment, the cyclic AMP agonist is forskolin. In another embodiment, the TGF-β inhibitor is RepSox. In another embodiment, the exogenous myogenic factor is MyoD.

The exact nature of the components configured in the inventive kit depends on its intended purpose. For example, some embodiments are configured for the purpose of generating iMPCs. In some embodiments, the kit is configured for generating iMPCs from somatic cells obtained from a sample. In yet other embodiments, the kit is configured to treat a subject in need thereof with iMPCs, e.g., including reagents necessary to maintain, expand and/or differentiate iMPCs to muscle. A kit can also contain a matrix or scaffold as described herein to support iMPCs or skeletal muscle differentiated therefrom. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to generate iMPCs from somatic cells and/or treat a subject in need thereof with iMPCs. Optionally, the kit also contains other useful components, such as, primers, diluents, buffers, pipetting or measuring tools or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in cell culture. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing the composition described above. The packaging material generally has an external label which indicates the contents and/or purpose of the kit and/or its components.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Kohler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

Some embodiments of the technology described herein can be defined according to any of the following numbered paragraphs:

1. A method for generating induced muscle progenitor cells (iMPCs), the method comprising: treating a population of somatic cells obtained from a subject with a cyclic AMP agonist, and a TGF-β inhibitor for a time and under conditions that induce dedifferentiation of the somatic cells to a population of cells comprising iMPCs.

2. The method of paragraph 1, wherein the somatic cells are fibroblasts.

3. The method of paragraph 2, further comprising expressing an exogenous myogenic factor in the somatic cells.

4. The method of paragraph 3, wherein the exogenous myogenic factor is MyoD.

5. The method of paragraph 4, wherein the exogenous MyoD is expressed transiently.

6. The method of paragraph 4, wherein the exogenous MyoD is expressed for a minimum of 2 days.

7. The method of paragraph 1, wherein the cyclic AMP agonist is forskolin.

8. The method of paragraph 7, wherein the concentration of forskolin is 1μM to 10μM, inclusive.

9. The method of paragraph 1, wherein the TGF-β inhibitor is RepSox, SB-431542 or ALK5 Inhibitor II.

10. The method of paragraph 1, wherein the TGF-β inhibitor is RepSox.

11. The method of paragraph 10, wherein the concentration of RepSox is 1μM to 10μM, inclusive.

12. The method of paragraph 1, wherein the somatic cells are muscle biopsy or muscle-derived explants and the iMPCs are muscle-induced iMPCs (M-iMPCs).

13. The method of paragraph 1, further comprising culturing the somatic cells and/or population of cells comprising iMPCs with ascorbic acid.

14. The method of paragraph 13, wherein the concentration of ascorbic acid is 20-100 μg/ml, inclusive.

15. The method of paragraph 1, further comprising culturing the somatic cells and/or population of cells comprising iMPCs with a GSK3β inhibitor.

16. The method of paragraph 15, wherein the GSK3β inhibitor is CHIR99021.

17. The method of paragraph 16, wherein the concentration of CHIR99021 is 1μM to 20μM, inclusive.

18. The method of paragraph 1, further comprising a step of isolating an iMPC and plating it as a clonal culture.

19. The method of paragraph 1, wherein the iMPCs are proliferative, self-renewing and capable of forming skeletal muscle myotubes.

20. The method of paragraph 1, wherein the iMPCs can be maintained in culture for at least 4 months.

21. The method of paragraph 20, wherein the iMPCs can be maintained in culture for greater than 6 months.

22. The method of paragraph 1, wherein the resulting cells do not comprise exogenous nucleic acid relative to the population of somatic cells.

23. The method of paragraph 1, wherein the population of cells is a heterogeneous culture of cells.

24. The method of paragraph 23, wherein the population of cells further comprises differentiated skeletal muscle cells.

25. The method of paragraph 1, wherein the dedifferentiation of the somatic cells to iMPCs does not go through a transient pluripotent state.

26. The method of paragraph 1, wherein the population express one or more of the following markers: Pax7, Myf5, Cxcr4, Myf6, VCAM1, Myog and MyHC.

27. The method of paragraph 26, wherein the iMPCs do not detectably express fibroblast markers.

28. The method of paragraph 27, wherein the fibroblast markers are Col5a1, Thy1, and Fbln5.

29. The method of paragraph 1, wherein the iMPCs are mononucleated.

30. An in vitro heterogeneous population of skeletal muscle cells comprising induced muscle progenitor cells (iMPCs).

31. The population of paragraph 30, wherein the iMPCs do not comprise exogenous nucleic acid encoding a MyoD transcription factor.

32. The in vitro heterogeneous population of skeletal muscle cells of paragraph 30, wherein the heterogeneous population can be maintained in culture without loss of phenotype for at least 6 months.

33. The in vitro heterogeneous population of skeletal muscle cells of paragraph 30, wherein the in vitro heterogeneous population is maintained in medium comprising ascorbic acid, GSK3β inhibitor and an FGF.

34. A composition comprising a population of iMPCs and a culture medium comprising ascorbic acid, a TGFβ inhibitor and a cyclic AMP agonist.

35. The composition of paragraph 34, wherein the concentration of ascorbic acid is

36. The composition of paragraph 34, wherein the TGFb inhibitor is RepSox, SB-431542 or ALK5 Inhibitor II.

37. The method of paragraph 36, wherein the TGF-β inhibitor is RepSox.

38. The method of paragraph 37, wherein the concentration of RepSox is 1μM to 10μM, inclusive.

39. The composition of paragraph 34, wherein the cAMP agonist is forskolin.

40. The composition of paragraph 39, wherein the concentration of forskolin is 1μM to 10μM, inclusive.

41. The composition of paragraph 34, wherein the ascorbic acid is 50 μg/ml, the TGFβ inhibitor is RepSox, at a concentration of 5μM, and the cAMP agonist is forskolin, at a concentration of 5μM.

42. The composition of paragraph 41, further comprising CHIR99021, at a concentration of 10 μM.

43. The composition of paragraph 41, further comprising bFGF at a concentration of 10 ng/ml.

44. A method for promoting muscle regeneration and/or repair, the method comprising: administering a therapeutically effective amount of iMPCs to a subject in need thereof.

45. The method of paragraph 44, wherein the iMPCs are prepared according to the method of paragraph 1.

46. The method of paragraph 44, wherein the iMPCs are autologous to the subject.

47. The method of paragraph 44, wherein the therapeutically effective amount comprises at least 1×10⁵ cells.

48. The method of paragraph 44, wherein the therapeutically effective amount comprises at least 1×10⁶ cells.

49. A method for treating a muscle disease or disorder, the method comprising: administering a therapeutically effective amount of iMPCs to a subject in need thereof.

50. The method of paragraph 49, wherein the iMPCs are prepared according to the method of paragraph 1.

51. The method of paragraph 49, wherein the iMPCs are autologous to the subject.

52. The method of paragraph 49, wherein the therapeutically effective amount comprises at least 1×10⁵ cells.

53. The method of paragraph 49, wherein the therapeutically effective amount comprises at least 1×10⁶ cells.

54. The method of paragraph 49, wherein the muscle disease or disorder is characterized by a gene mutation and/or deficiency.

55. A method of screening for a drug useful in the treatment of a disease comprising;
obtaining a sample from a subject with the disease;
generating iMPCs by the method of paragraph 1;
contacting the iMPCs generated with a drug, and;
determining the effect of the drug on said cells.

56. The method of paragraph 55, wherein the disease is characterized by a gene mutation and/or deficiency.

57. The method of paragraph 55, wherein the disease is muscle-associated disorder.

58. The method of paragraph 57, wherein the muscle-associated disorder is duchenne muscular dystrophy, becker muscular dystrophy, facioscapulohumeral muscular dystrophy, and myotonic dystrophy, congenital muscular dystrophy, distal muscular dystrophy, emery-dreifuss muscular dystrophy, oculopharyngeal muscular dystrophy, limb girdle muscular dystrophy.

59. The method of paragraph 55, wherein the drug is a known or experimental drug.

60. The method of paragraph 59, wherein a combination of drugs is screened.

61. The method of paragraph 55, wherein the drug is beneficial if an increase in the mutated gene's expression is observed and the drug is not beneficial if a decrease or no change in the mutated gene's expression is observed.

62. The method of paragraph 58, wherein the drug is beneficial if there is an increase in muscle regeneration and/or repair and the drug is not beneficial if there is a decrease or no change in muscle regeneration and/or repair.

63. The method of paragraph 55, further comprising administering the drug screened that has been determined to be beneficial to the subject with the disease.

64. A composition comprising a cAMP agonist, a TGFβ inhibitor and ascorbic acid.

65. The composition of paragraph 64, wherein the c-AMP agonist is forskolin.

66. The composition of paragraph 65, wherein the forskolin is present at a concentration of 1 μM to 10 μM, inclusive.

67. The composition of paragraph 64, wherein the TGFβ inhibitor is RepSox, SB-431542 or ALK5 Inhibitor II.

68. The composition of paragraph 67, wherein the TGFβ inhibitor is RepSox.

69. The composition of paragraph 68, wherein the RepSox is present at a composition of 1 μM to 10 μM, inclusive.

70. The composition of paragraph 64, wherein the acsorbic acid is present at a concentration of 20-100 μg/ml, inclusive.

71. The composition of paragraph 64, further comprising a GSK3β inhibitor.

72. The composition of paragraph 71, wherein the GSK3β inhibitor is CHIR99021.

73. The composition of paragraph 72, wherein the CHIR99021 is present at a concentration of 1 mM to 20 μM, inclusive.

74. The composition of paragraph 64, further comprising bFGF.

75. The composition of paragraph 74, wherein the concentration of bFGF is 1 ng/ml to 20 ng/ml, inclusive.

76. The composition of any one of paragraphs 64 to 75, for use in generating or maintaining a population of iMPCs.

77. The composition of any one of paragraphs 64-76, wherein the concentration or amount of the cAMP agonist, TGFβ inhibitor, ascorbic acid, and if present, GSK3β inhibitor and bFGF are each present at the same multiple, greater than 10 times, of the concentration or amount used in culture medium to induce or maintain a population of iPMCs.

78. The composition of paragraph 77, wherein the multiple of the concentration or amount used in culture medium is at least 100.

79. The composition of paragraph 77, wherein the multiple of the concentration or amount used in culture medium is at least 1000.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

We report a method to reprogram or dedifferentiate mouse fibroblasts into myogenic cells with characteristics of muscle stem and progenitor cells. Overexpression of the myogenic transcription factor MyoD in fibroblasts has previously been shown to induce direct conversion of these differentiated cell types into myogenic cells. Muscle-like cells generated with this approach are also differentiated (i.e. postmitotic) and therefore cannot be further propagated, limiting their usefulness in a potential therapeutic setting.

Here, we show that transient expression of MyoD combined with exposure to a cocktail of small molecules (Ascorbate, TGFβ inhibitor, Forskolin, bFGF), generates a population of small proliferative cells that express markers of muscle stem cells (e.g., Pax3, Pax7 and Myf5), can be propagated indefinitely in culture, give rise to multinucleated myofibers, and exhibit spontaneous contractions; indicating their self-renewal and differentiation potential. We call these cells induced muscle stem cells (iMPCs). We can also obtain these cells from adult tail tip friboblasts. Moreover, we recovered an iMPC clone at low efficiency even in the absence of transgenic MyoD expression, indicating that the chemicals alone are sufficient to induce dedifferentiation of skin cells to iMPCs.

To efficiently induce a myogenic stem cell program in fibroblasts, cells are infected with a dox-inducible MyoD overexpression vector in KO-DMEM medium supplemented with 10% FBS, 10% Serum Replacement (containing ascorbate), 1% GlutaMAX, 1% non-essential amino acids, 1% penicillin-streptomycin, 0.5%-mercaptoethanol, 5 μM Forskolin, 5 μM TGFβ inhibitor RepSox and 10 ng/ml bFGF. Once muscle stem cell clones appear, dox is removed from the media and cultures are propagated in chemicals alone, which is critical for the maintenance of self-renewal. Contractility and Pax7 positivity are observed as early as 4 days following treatment with MyoD overexpression. To generate chemically-induced myogenic cultures, MyoD overexpression is omitted but cultures are otherwise treated equally. Thus far, cell cultures have been passaged for over 6 months while maintaining the potential for self-renewal and differentiation into contracting myofibers.

The inventors demonstrate a method of generating expandable myogenic cells from skin cells (i.e. fibroblasts), which can be reprogrammed using a combination of MyoD expression and small molecules, directly into self-renewing, functional muscle stem-like cells (i.e. contractions). While demonstrated herein using skin-derived fibroblasts (and adult muscle-derived cells; see below), other somatic cells are also candidates for dedifferentiation to a myogenic progenitor phenotype using this approach. The resulting cells can also serve as a platform to study diseases in vitro or to introduce or repair mutations relevant in muscle biology or muscle disease. The chemicals used in this method can also facilitate the expansion of stem cells isolated directly from muscle tissue, which is impossible with current protocols. The methods described herein can be used to derive patient-specific muscle cells for the treatment of, e.g., degenerative diseases, and have implications for regenerative medicine, disease modeling and drug screening approaches.

### Example 2

MyoD and Small Molecules Induce Progenitor-Like Cells from Fibroblasts

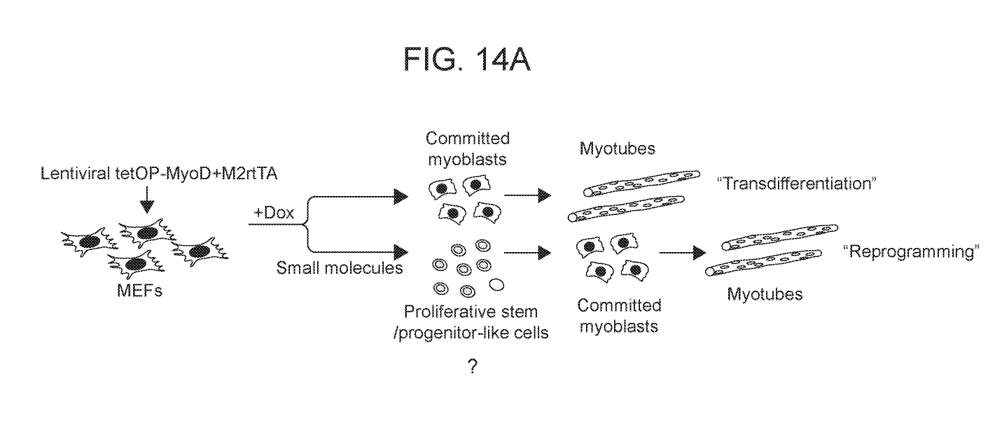

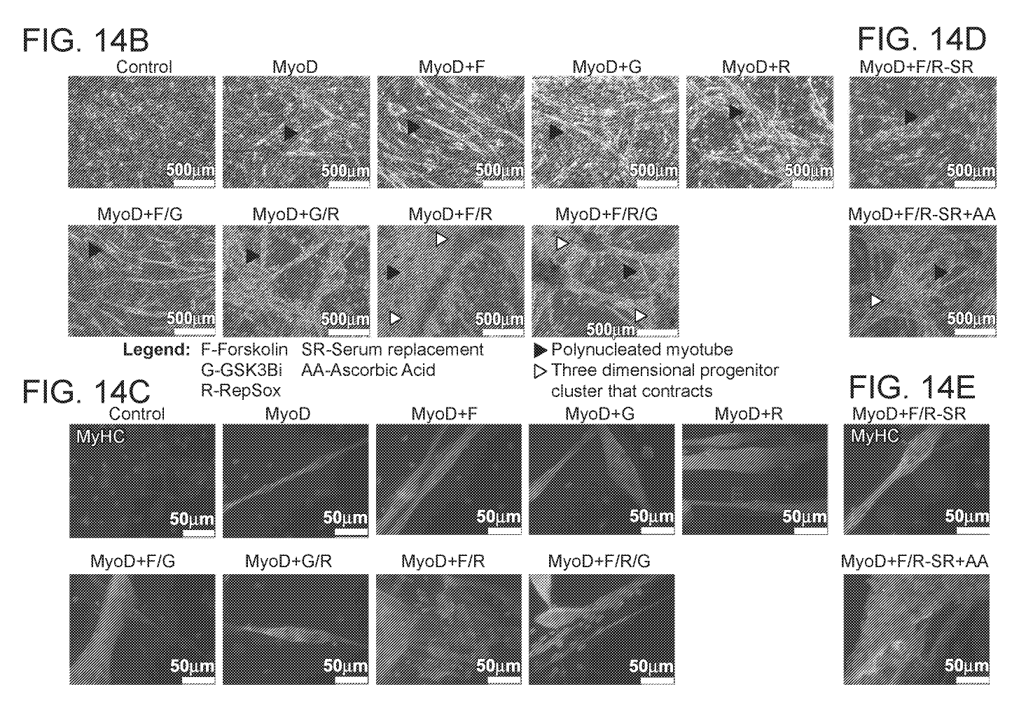

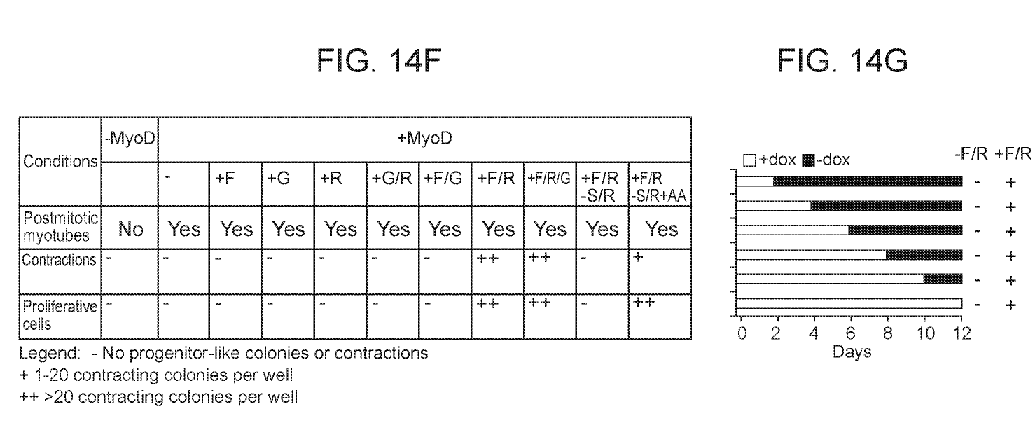

To induce conversion of fibroblasts into cells of the skeletal muscle lineage, MyoD, which has previously been shown to trigger transdifferentiation of different somatic cell types into post-mitotic myotubes was over expressed (FIG. 14A, top row). A doxycycline (dox) inducible lentiviral system (tetOP-MyoD) was engineereed, allowing for inducible and reversible activation of MyoD in target cells. Mouse embryonic fibroblasts (MEFs) transduced with lentiviral vectors co-expressing tetOP-MyoD and M2rtTA24 showed extensive nuclear staining for the MyoD protein after 24 hours of dox administration (FIG. 7), and gave rise to elongated myotubes after 24-96 hours of dox exposure (FIG. 14B). The myogenic identity of converted cells was confirmed by immunofluorescence staining for the differentiation marker myosin heavy chain (MyHC), which was absent in uninfected MEFs (FIG. 14C).

MEFs undergoing MyoD-induced lineage conversion were exposed to various small molecules and cytokines in an attempt to induce reprogramming into a proliferative, myogenic progenitor-like cell state in addition to mature myotubes (FIG. 14A, bottom row). Specifically, compounds including the GSK3β inhibitor CHIR99021 (abbreviated as “G”) and the TGF-β1 receptor inhibitor RepSox (abbreviated as “R”) were used. In addition, the effect of the cyclic AMP agonist Forskolin (abbreviated as “F”), which reportedly facilitates the transient expansion of satellite cells in vitro was tested. These compounds were added individually or combinatorially to MEFs expressing MyoD and cultured in Knockout DMEM media containing 10% KOSR and 10% FBS and supplemented with 10 ng/ml bFGF, which promotes satellite cell and myoblast growth. Individual treatment of MyoD-expressing MEFs with F, G or R as well as combinatorial treatment with G/R or F/G increased the formation of MyHC⁺, polynucleated myotubes but did not generate any proliferative cells (FIGS. 14B and 14C). By contrast, F/R treatment led to a proportional increase of myotubes and proliferative cells, indicative of induction of a progenitor-like cell population, which subsequently differentiated into polynucleated myotubes. F/R/G treatment did not show any further increase in the number of proliferative cells or myotubes compared to F/R treatment alone and therefore the GSK3β inhibitor CHIR99021 was omitted from the dedifferentiation culture conditions. Strikingly, spontaneous and robust contractions within F/R treated cultures were observed, which were never detected in MEFs expressing MyoD alone (Live video monitoring was used to demonstrate that cells contract). Cultures treated with these small molecules typically assembled into three-dimensional colonies with emanating myotubes and numerous small, round cells that seem to be muscle progenitors (FIG. 14B and live video monitoring was used to monitor the muscle progenitors). KOSR media contains ascorbic acid, which enhances reprogramming into iPSCs by modulating epigenetic regulators. To determine whether ascorbic acid is required for the induction of these proliferative, putative progenitor cells using our culture system, MEFs expressing MyoD were exposed to F/R in the presence or absence of ascorbic acid using DMEM media containing FBS and bFGF but lacking KOSR. While post-mitotic myotubes readily formed in the absence of ascorbic acid, the generation of proliferative cells and contracting myotubes depended on ascorbic acid, emphasizing the importance of this vitamin in the generation of the progenitor-like cell population (FIGS. 14D and 14E). A summary of the various conditions and their effect on proliferation, differentiation and contraction is provided in FIG. 14F.

To assess the temporal requirement for exogenous MyoD expression and small molecules during the establishment and maintenance of our progenitor-like cell population, infected MEFs were treated with dox for 0, 2, 4, 6, 8 or 10 days in the presence of F/R, followed by 5 days of dox withdrawal before scoring for colonies that proliferated and contracted (FIG. 14G). It was found that as little as 2 days of exogenous MyoD induction was sufficient to generate proliferative cultures containing contracting myotubes. By contrast, continuous exposure of cultures to F/R was required to maintain these cultures upon passaging (FIG. 14G and data not shown). Thus, without being bound by any particular theory, small molecules and growth factors collaborate with transient MyoD expression to endow MEFs with a myogenic progenitor-like state. These cells are provisionally termed “induced myogenic progenitor cells” (iMPCs).

iMPCs Self-Renew and Express Myogenic Stem and Progenitor Cell Markers

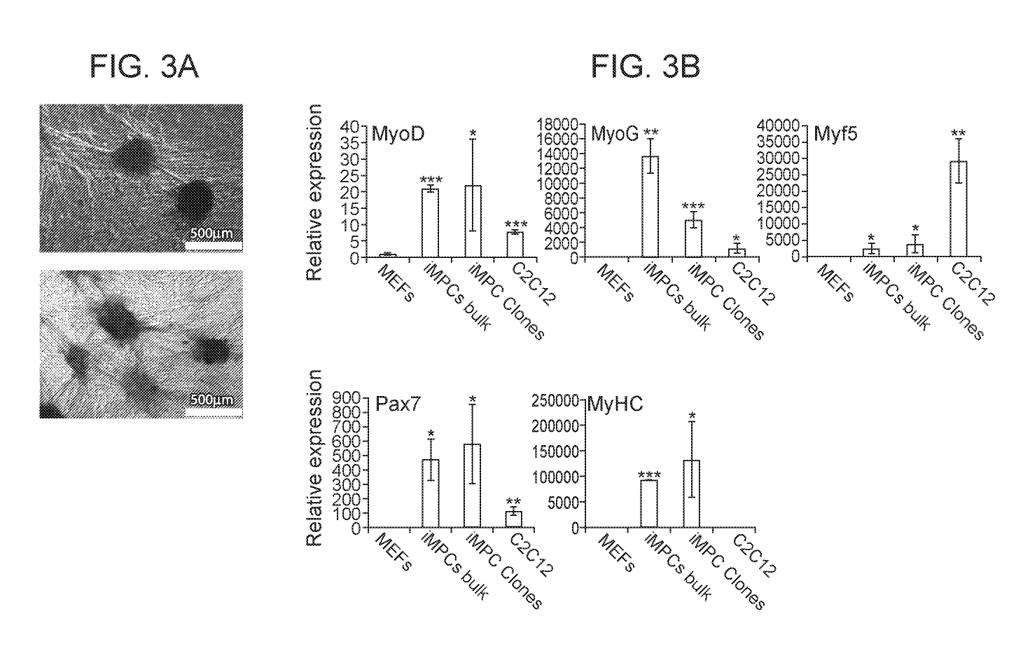

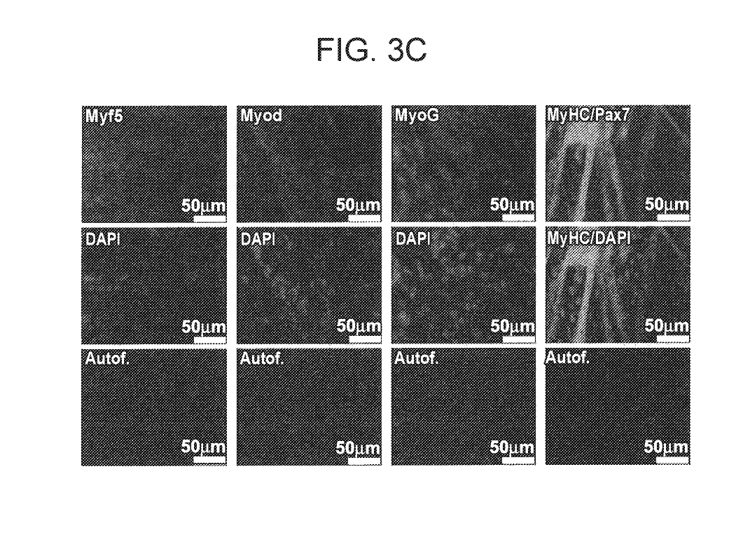

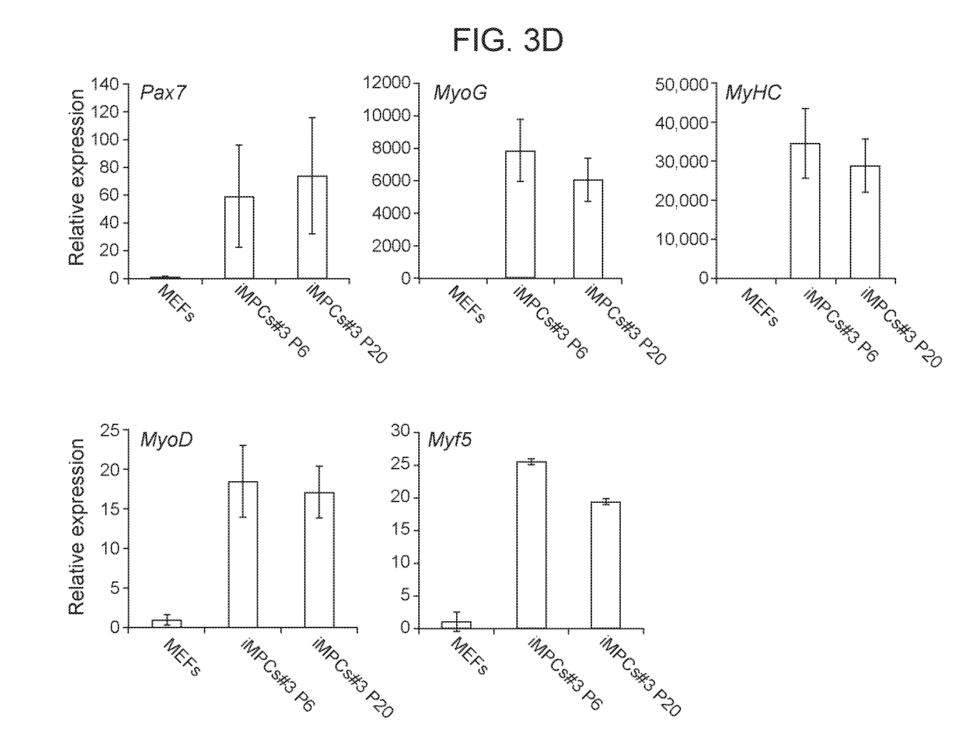

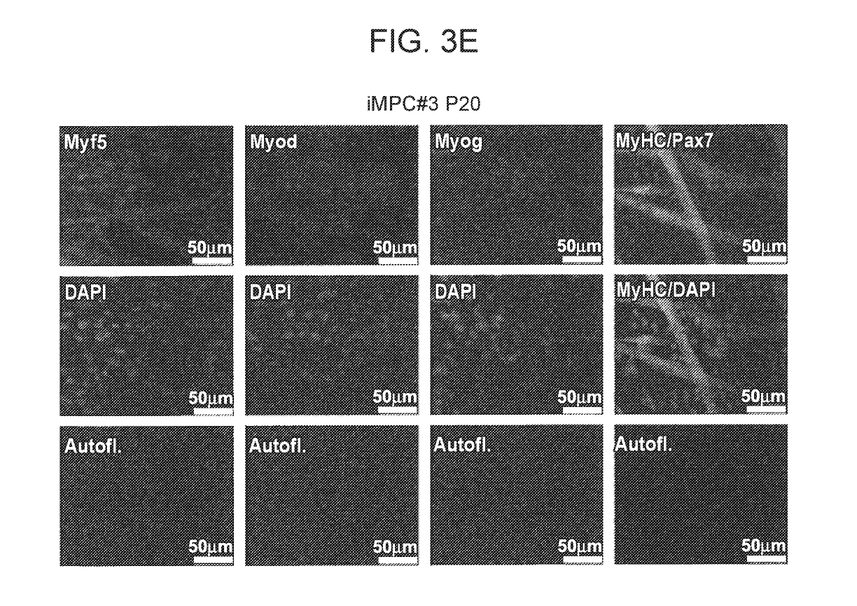

Continuous self-renewal and differentiation are hallmarks of stem cells. To determine whether iMPCs meet these criteria, either bulk cultures or clonal derivatives isolated from three-dimensional colonies were repeatedly passaged. Both types of cultures continued to grow for at least 6 months or 20-24 passages while retaining the potential to produce contracting myotubes, indicating prolonged self-renewal potential in the presence of appropriate growth factors and small molecules (FIG. 3A). Mononucleated cells isolated from either bulk or clonal iMPC lines always formed heterogeneous cultures containing both proliferative cells as well as polynucleated myotubes upon replating (data not shown). Without being bound by any particular theory, this observation suggests that differentiated cells may assist to maintain progenitors in an undifferentiated state.

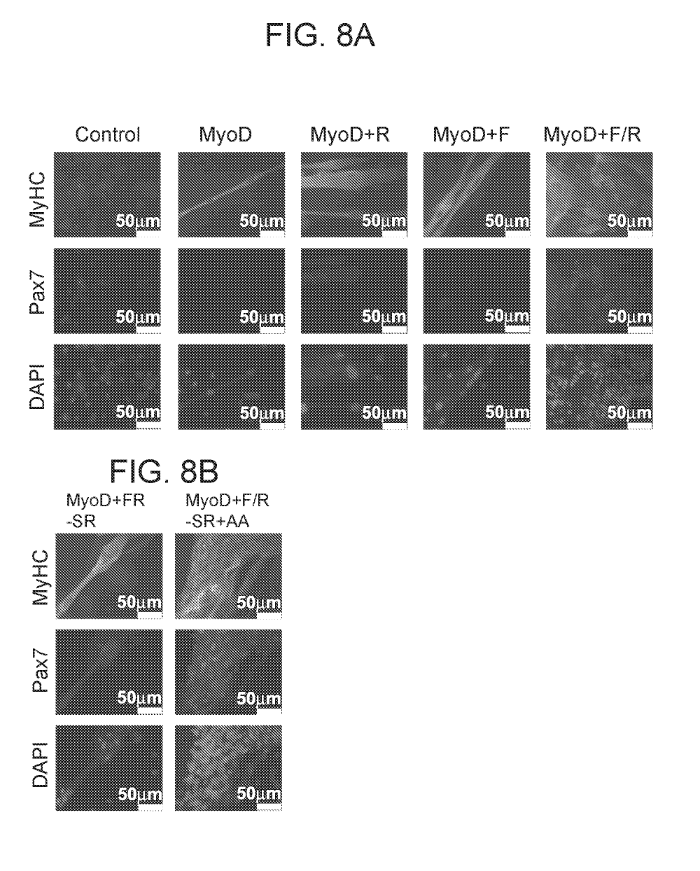

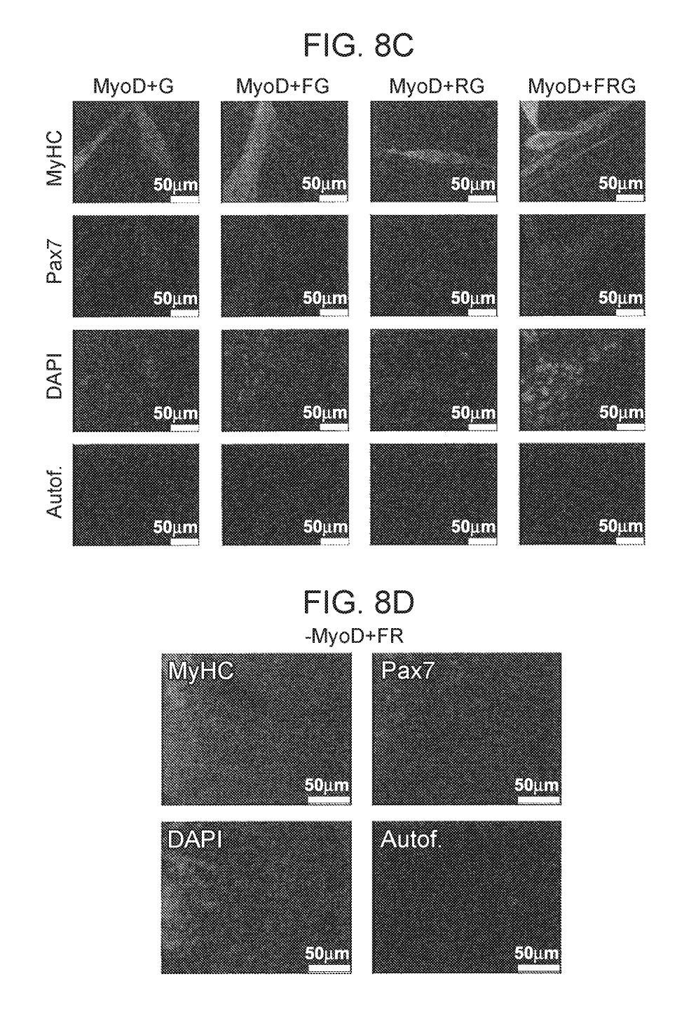

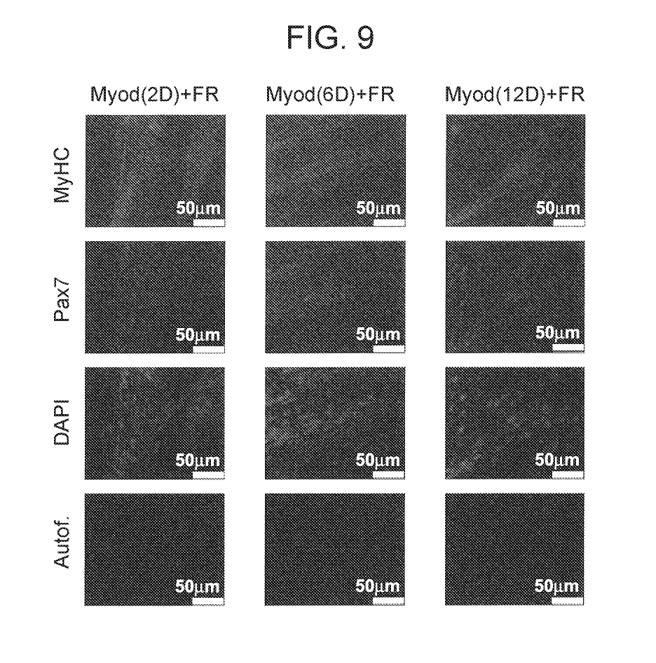

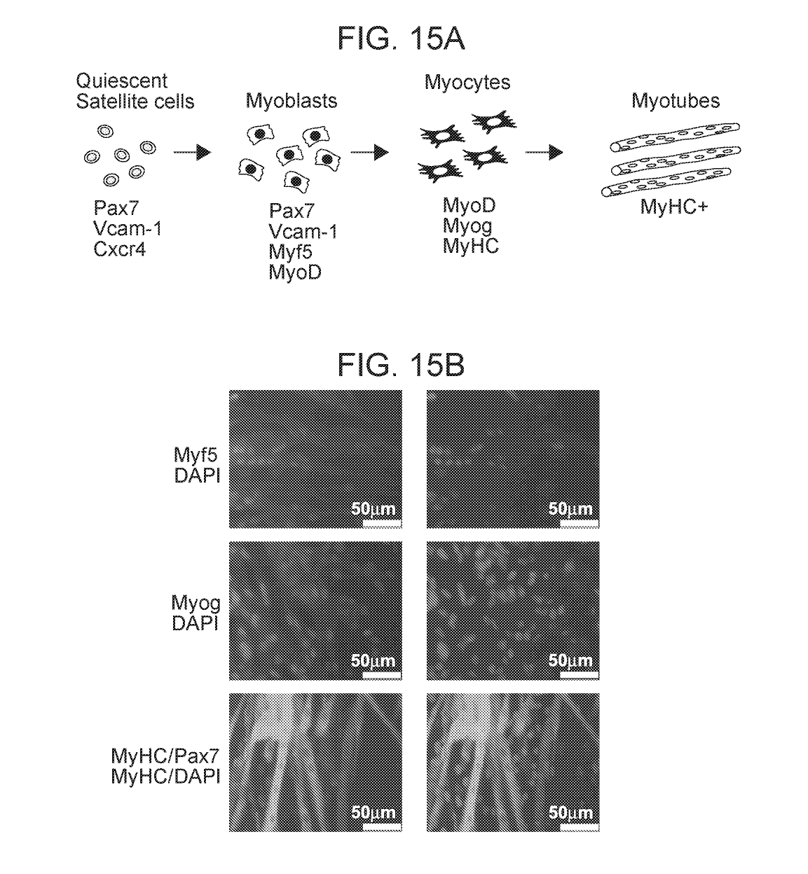

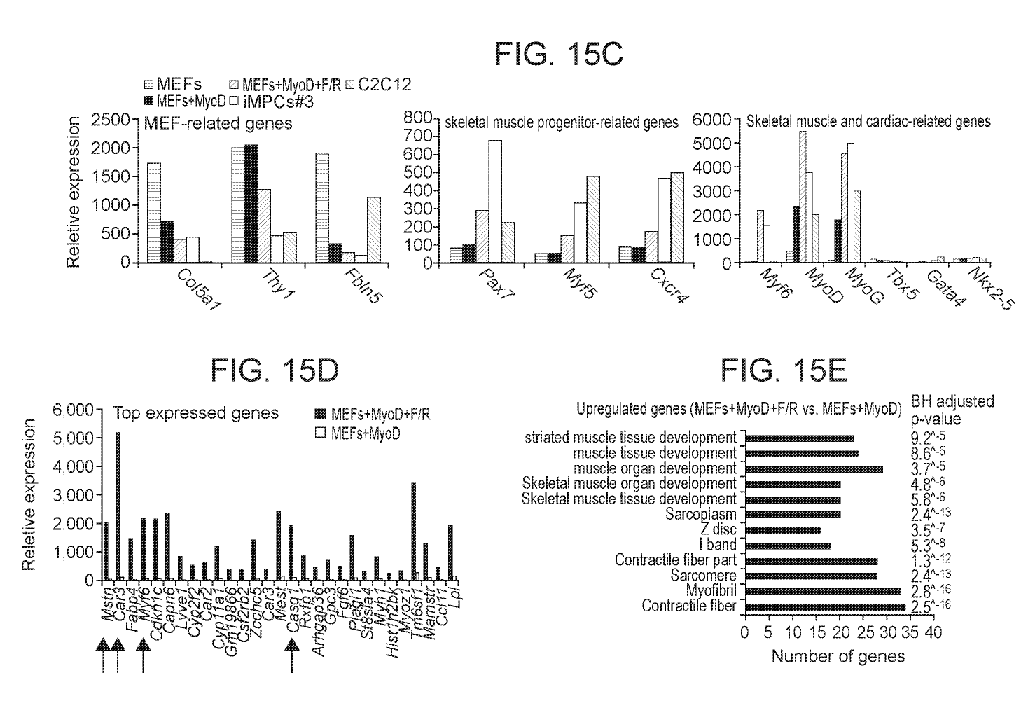

RT-qPCR and immunofluorescence were then performed to assess whether iMPC subsets express markers associated with different stages of myogenesis (see FIG. 15 for scheme). Clonal iMPC cultures at both low and high passage were found to contain cells that express the satellite cell marker Pax7, the myoblast marker Myf5 as well as the differentiation markers MyHC and Myog (FIGS. 3D and 15B). Importantly, Pax7⁺ cells were only detected in MEFs expressing MyoD and exposed to F/R in the presence of either KOSR or ascorbate, underscoring the importance of this small molecule combination for the activation of the Pax7 locus (FIGS. 8A and 8B). In addition, Pax7⁺ cells generally lacked MyHC expression, supporting the notion that these cells resemble undifferentiated satellite cells or myoblasts (FIG. 15B). Moreover, the iMPC cultures activated the endogenous MyoD locus, confirming earlier observation, described herein, that exogenous MyoD expression is only required for the initiation but not maintenance of proliferative progenitor cells (FIGS. 14G and 3D).

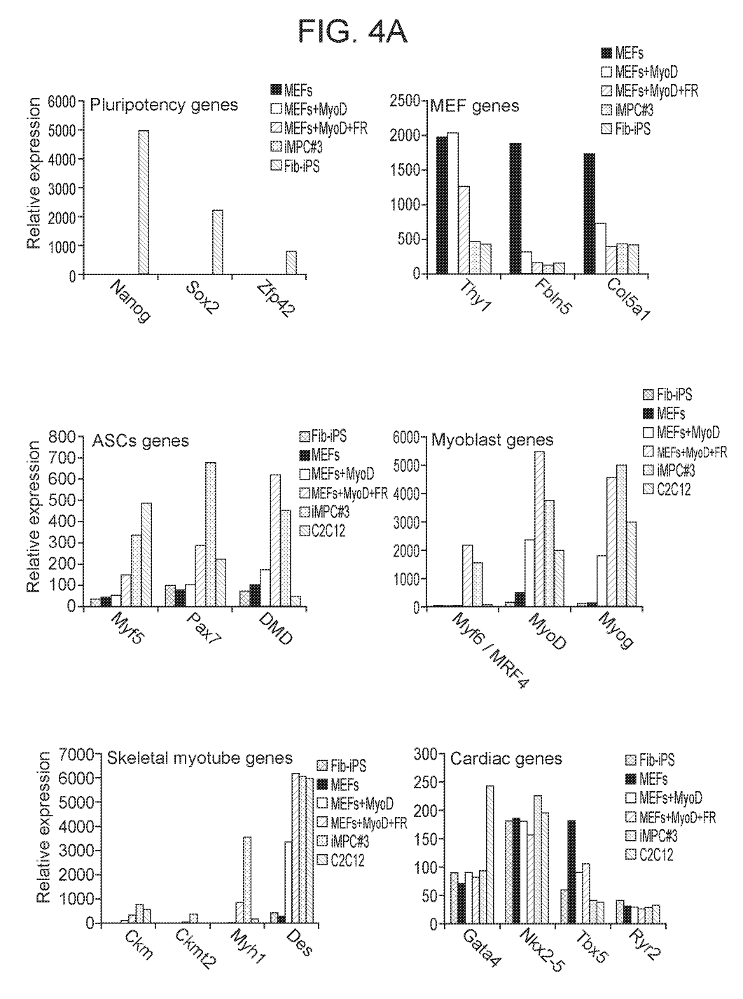

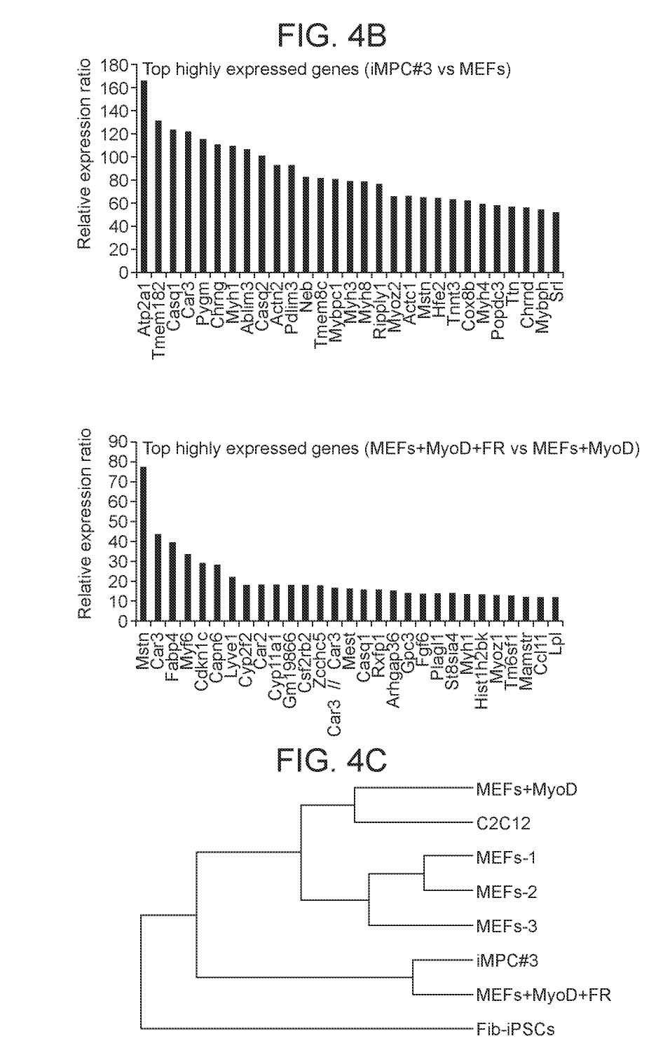

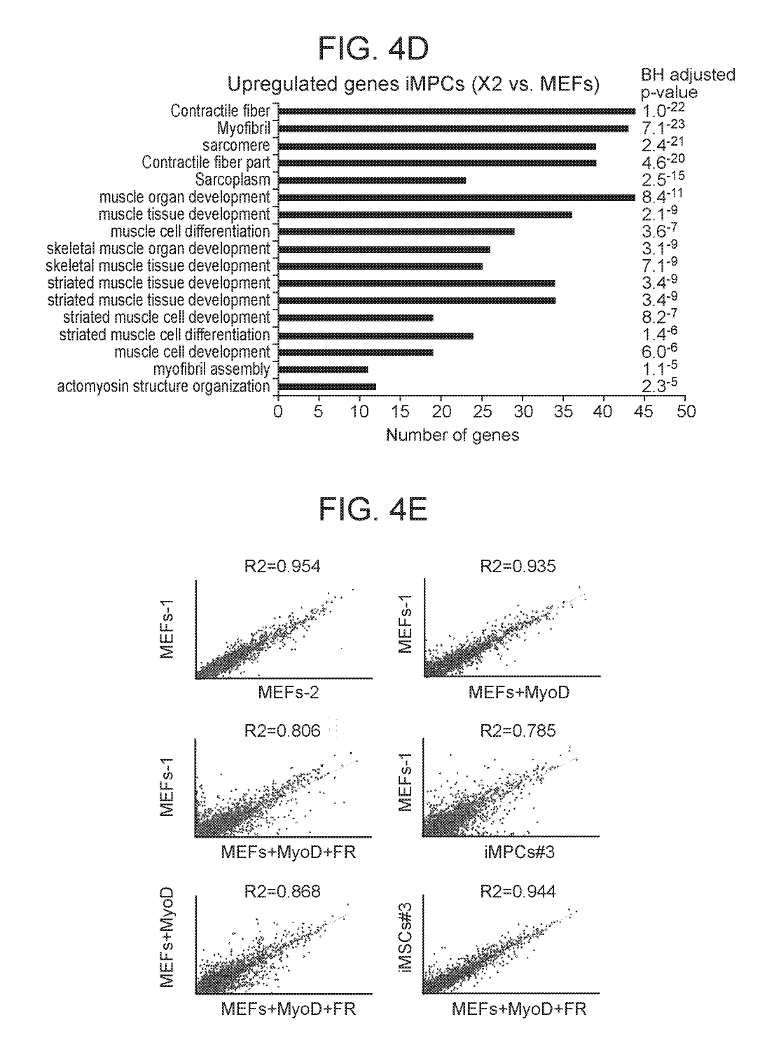

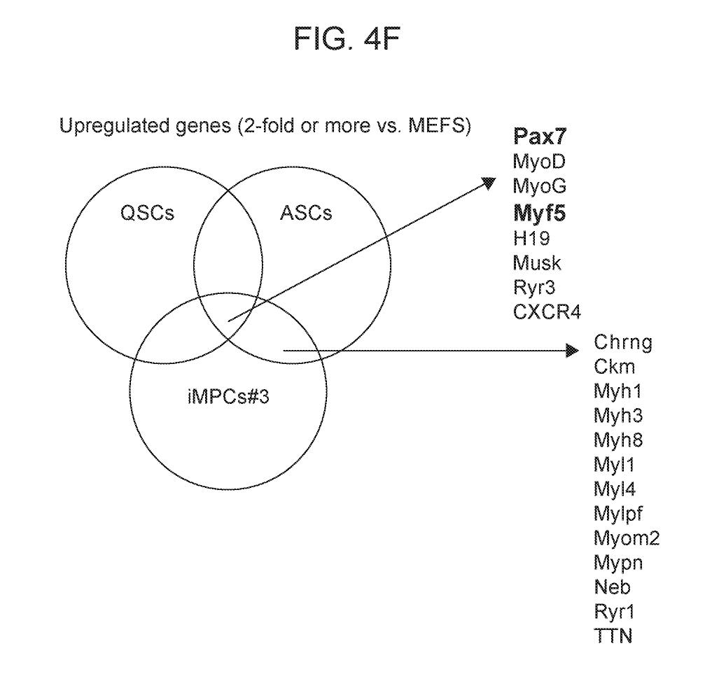

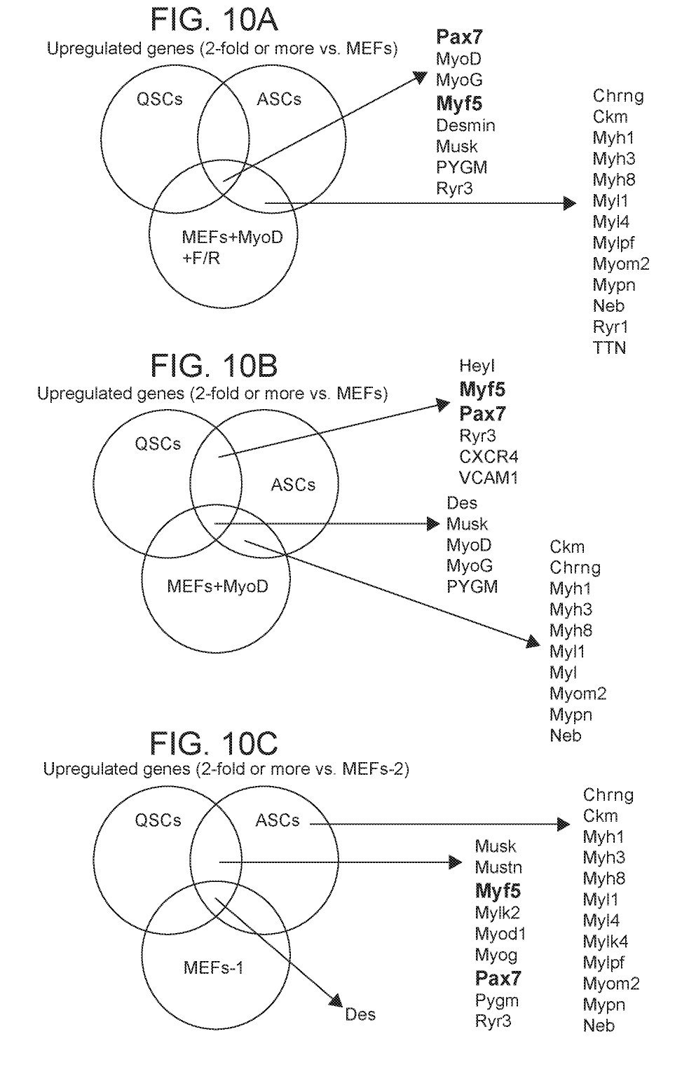

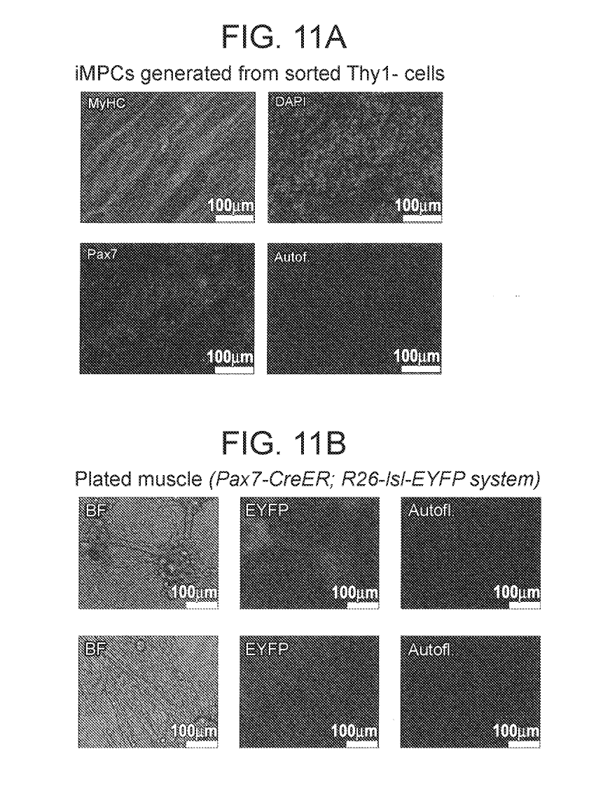

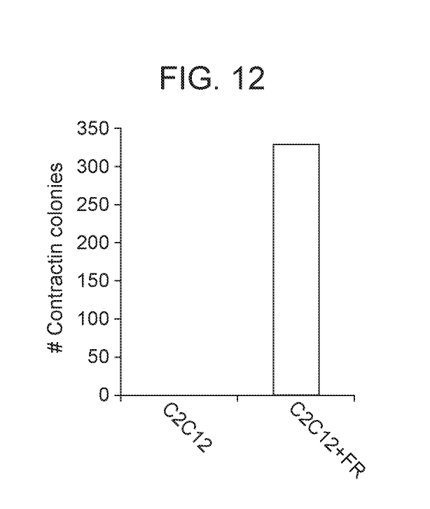

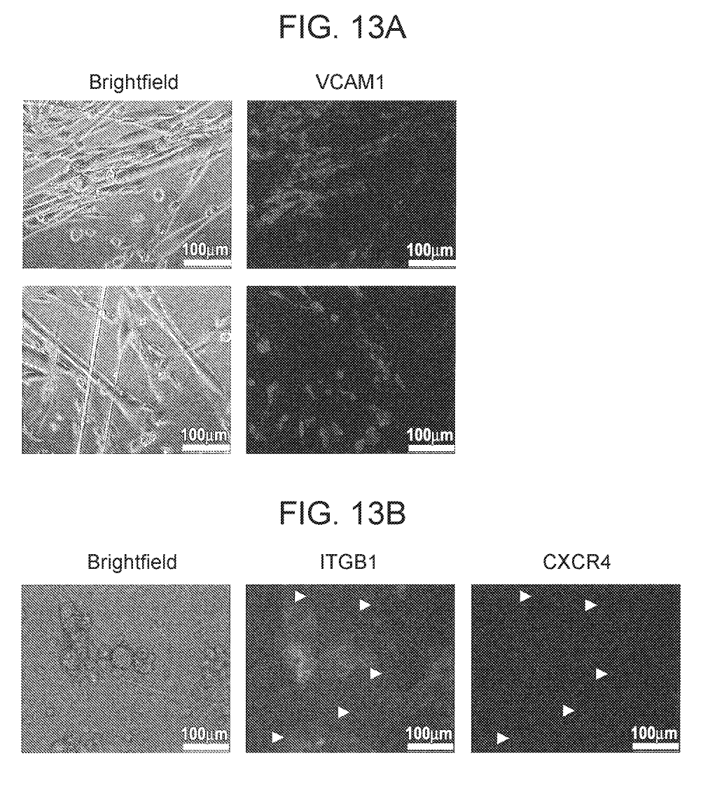

To compare the effects of MyoD expression and small molecule treatment on global transcriptional patterns, microarray analysis of (i) untreated MEFs, (ii) MEFs expressing MyoD alone (MEFs+MyoD), (iii) MEFs+MyoD in combination with F/R (MEFs+MyoD+F/R) for 14 days, (iv) a clonal iMPC line and (v) the immortalized myoblast cell line C2C12, was performed. Analysis of these samples revealed that fibroblast-associated genes such as Col5a1, Thy1 and Fbln5 were effectively downregulated in iMPCs and MEFs+MyoD+F/R and to a lesser extent in MEFs+MyoD (FIG. 15C, left). The inability of ectopic MyoD expression alone to effectively silence MEF genes is consistent with the previous observation that MyoD activates rather than represses genes. Importantly, genes associated with satellite cells (e.g., Pax7) and myoblasts (e.g., Myf5) were specifically upregulated in iMPCs and MEFs+MyoD+F/R but not in MEFs+MyoD, confirming that small molecules are critical for the activation of a myogenic stem and progenitor-like program (FIG. 15C, center and FIG. 4E). Notably, iMPCs expressed the progenitor cell-associated genes Myf5 and Cxcr4 at similar levels as the C2C12 myoblast cell line. A robust upregulation of genes associated with mature muscle tissue including Myf6 and Myog was observed in iMPC cultures, whereas markers associated with alternative lineages such as cardiomyocytes (e.g. Tbx5, Gata4, Nkx2-5) were not expressed at appreciable levels (FIG. 15C, right). A comparison of the top-expressed genes and associated gene ontology (GO) categories between MEFs undergoing canonical MyoD2-induced transdifferentiation (MEFs+MyoD) and those undergoing reprogramming (MEFs+MyoD+F/R) corroborated the conclusion that iMPC cultures represent skeletal muscle and not cardiac muscle (FIGS. 15D, 15E, FIG. 4F and FIG. 10A-10C). These results show that iMPC cultures contain skeletal muscle progenitors, which self-renew, express markers of satellite cells and myoblasts and undergo spontaneous differentiation into functional myotubes.

iMPCs Originate from Fibroblasts and do not Pass Through an iPSC State

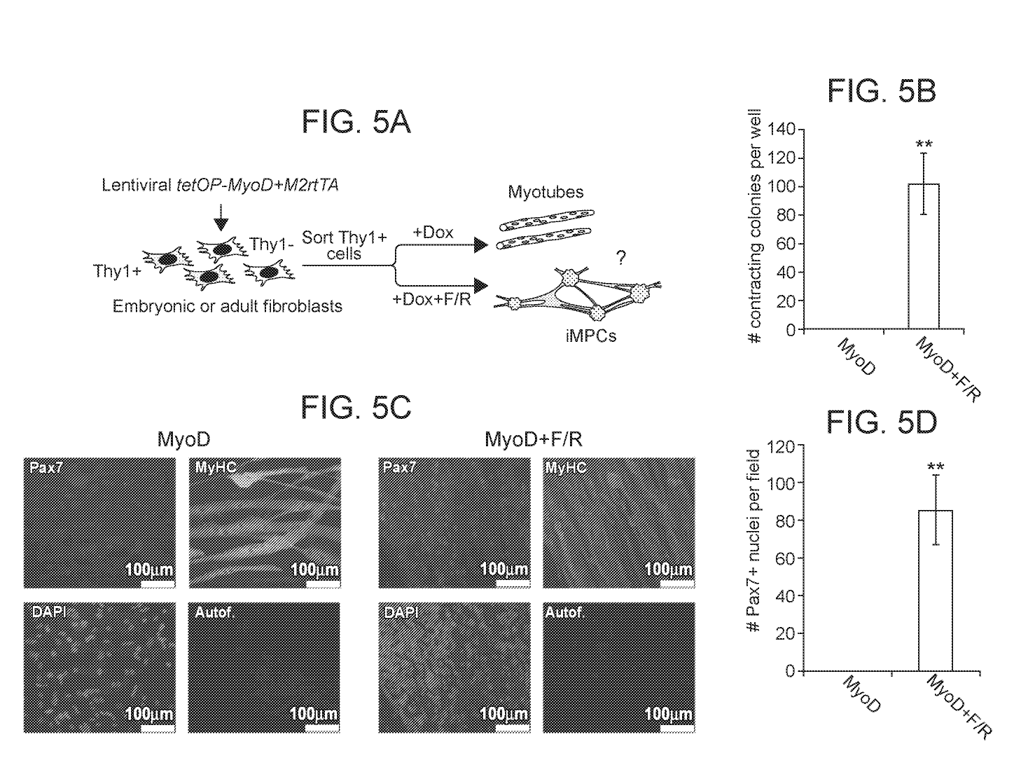

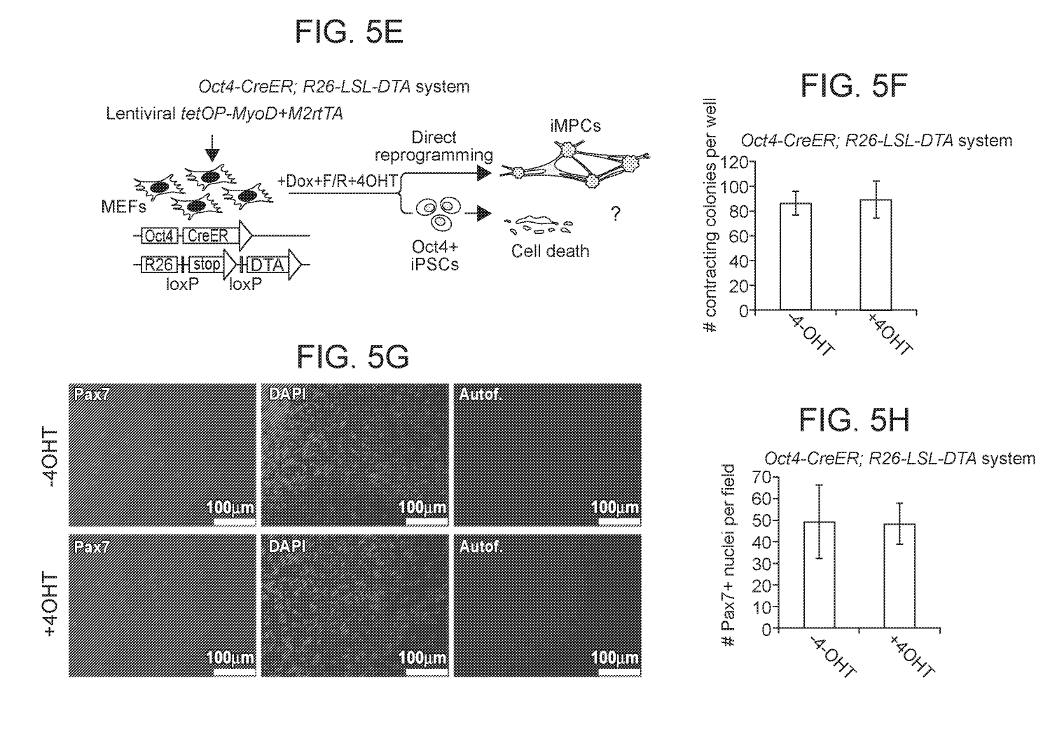

MEFs are a heterogeneous cell population comprised of mesenchymal, endothelial and several other cell types. To exclude the possibility that MyoD expression and small molecule treatment amplifies a pre-existing myogenic progenitor cell type present in the cultures, sorted MEFs and adult tail tip fibroblasts (TTFs) were sorted based on the fibroblast-associated marker Thy1 before inducing reprogramming with MyoD and small molecules (FIG. 5A). Proliferative and contracting iMPC cultures developed from both Thy1⁺ MEFs and TTFs upon overexpression of MyoD and treatment with F/R (FIG. 5B-D). By contrast, only post-mitotic myofibers emerged from these cell types upon overexpression of MyoD alone (FIG. 5B-D). Consistent with this finding, MyHC⁺ cells were detectable under both reprogramming and lineage conversion conditions, respectively, whereas Pax7⁺ cells were exclusively detected in cells undergoing reprogramming towards iMPCs (FIG. 5B-D).

As the small molecules used can also promote reprogramming of MEFs into iPSCs, it was critical to rule out that iMPC generation involves transient passage through an iPSC state, which can occur in transdifferentiation paradigms utilizing pluripotency-associated factors. MEFs carrying the pluripotency-specific Oct4-CreER allele was employed, in combination with the ROSA26-LSLDTA allele (FIG. 5E). Previous studies by the inventors had shown that activation of the endogenous Oct4 locus ablates pluripotent cells in the presence of 4-OHT using this system. MEFs harboring these alleles were infected with tetOP-MyoD and M2rtTA lentiviral vectors and lineage reprogramming was induced by adding dox and F/R in the presence or absence of 4-OHT. We recovered contractile, Pax7⁺ iMPC cultures at similar frequency using both conditions (FIG. 5F-5H). These data indicate that both embryonic and adult Thy1⁺ fibroblasts are amenable to lineage reprogramming into iMPCs and that this process does not involve passage through a transient pluripotent cell state.

iMPCs Engraft and Differentiate into Dystrophin+ Myofibers in Mdx Mice

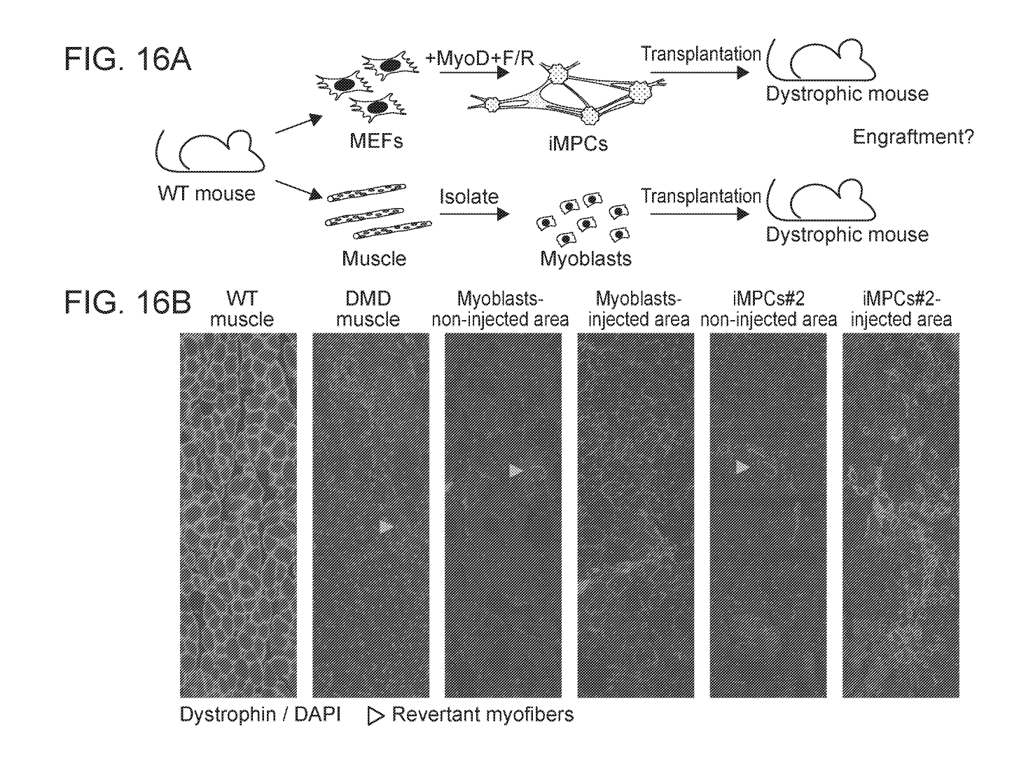

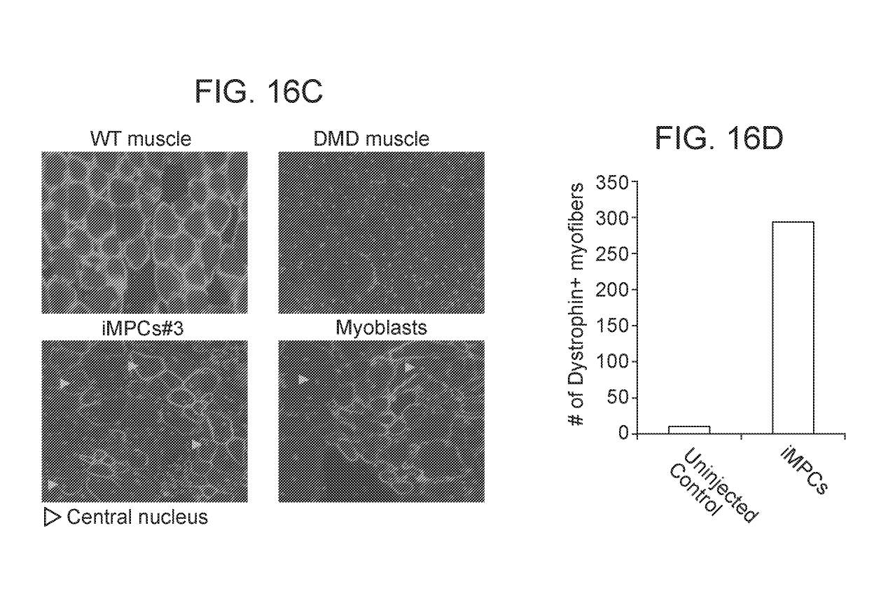

Skeletal muscle-derived stem and progenitor cells have the potential to contribute to muscle regeneration upon transplantation into dystrophic hosts. This represents a crucial functional assay to confirm the engraftment and differentiation potential of cells. To assess whether the reprogrammed cells meet this criterion, clonal iMPC lines were derived from MEFs using the aforementioned conditions and transplanted 1×10⁶ cells into the tibialis anterior (TA) or gastrocnemius of 12 week-old homozygous mdx dystrophic mice (FIG. 16A); mdx mice carry a spontaneous mutation within the dystrophin gene, which models Duchenne/Becker muscular dystrophy and causes muscle degeneration, thus providing a useful system for cell transplantation. In parallel, myoblasts were isolated from the muscles of Pax7-CreER; ROSA26-LSL-EYFP mice treated with tamoxifen. These cells were cultured in the presence of bFGF for 7 days and used as positive control for our transplantation experiments (FIG. 16A). While rare Dystrophin⁺ revertant myofibers in uninjected mdx muscle were detected, contiguous areas of Dystrophin⁺ myofibers in mdx muscle transplanted with either myoblasts or iMPCs were consistently observed (FIG. 16B). Dystophin⁺ myofibers derived from myoblasts or iMPCs had centrally located nuclei and varied in size, which is indicative of an active regenerative process involving both new fiber formation and repair of the damaged endogenous myofibers (FIGS. 16C and 16D). Altogether, these results demonstrate that iMPCs exhibit not only molecular but also key functional attributes of bona fide skeletal muscle progenitors.

iMPC Subsets are Hierarchically Connected and Recapitulate Myogenesis

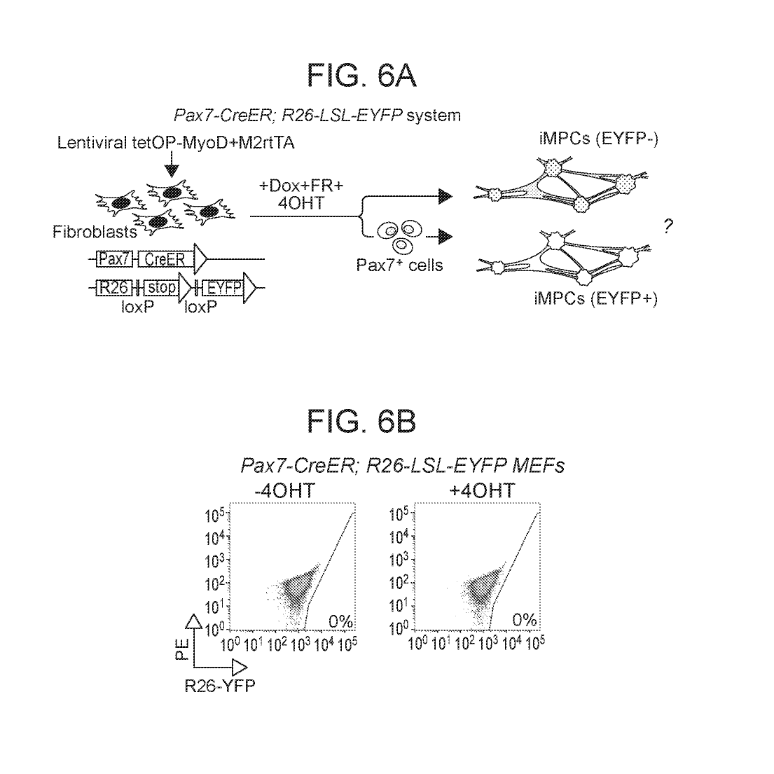

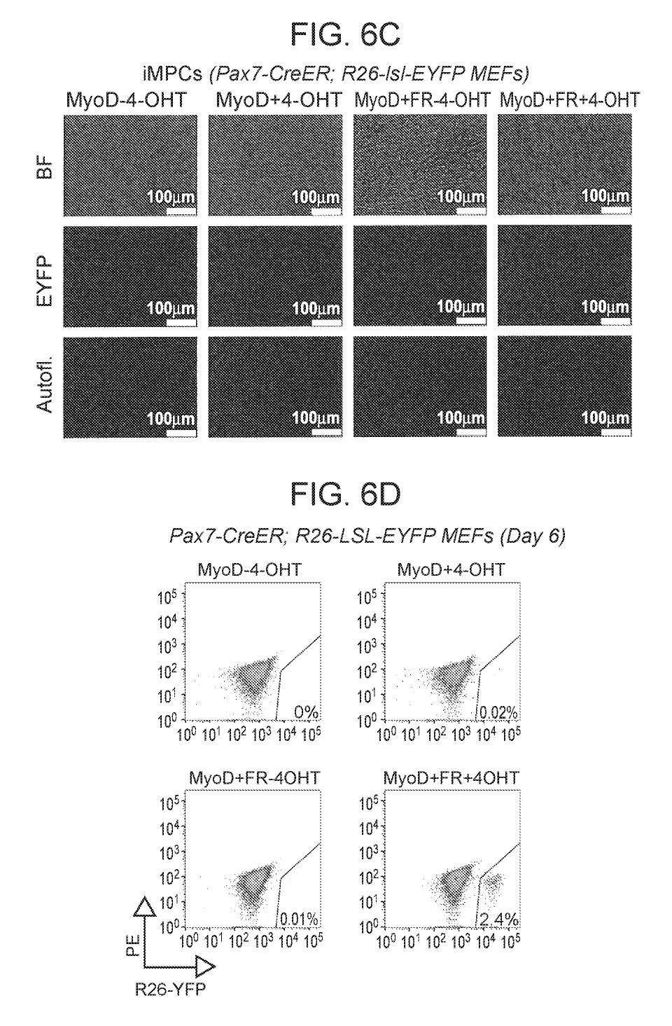

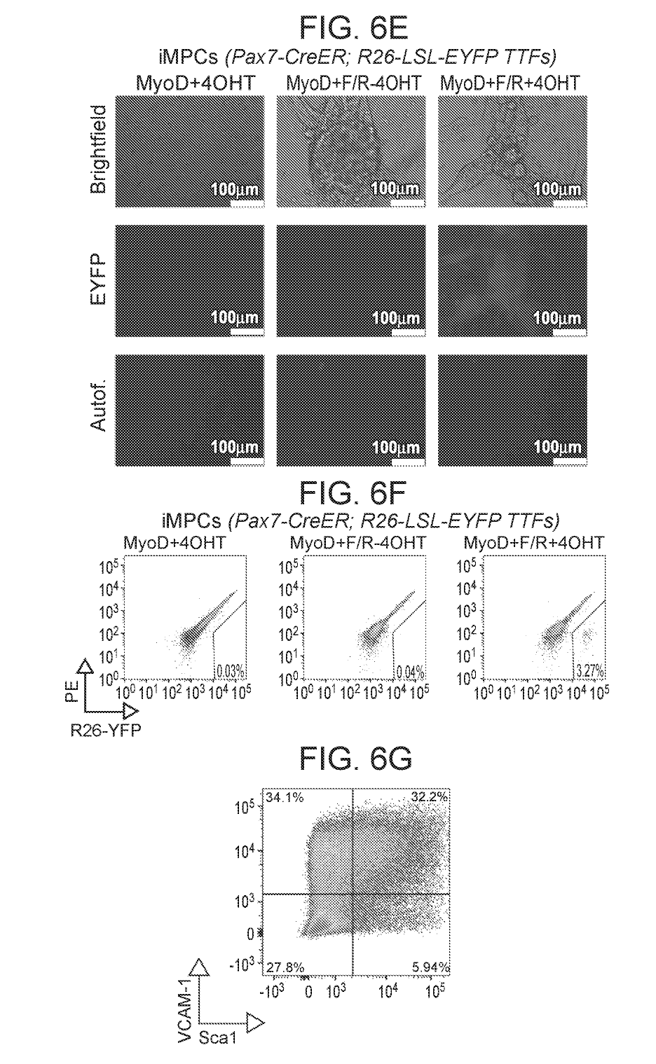

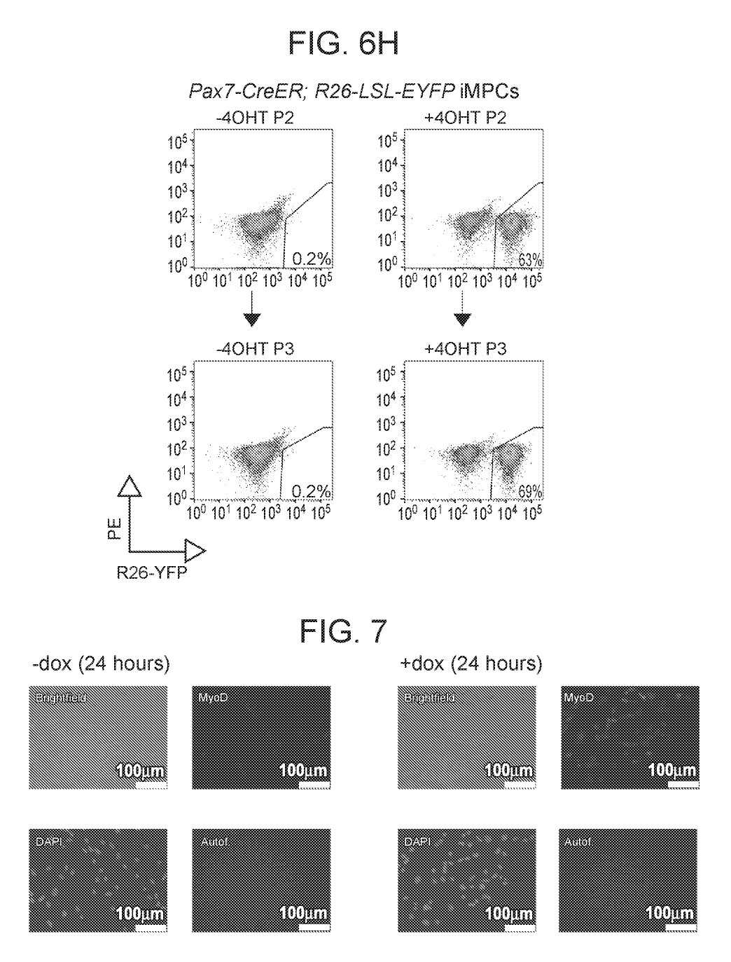

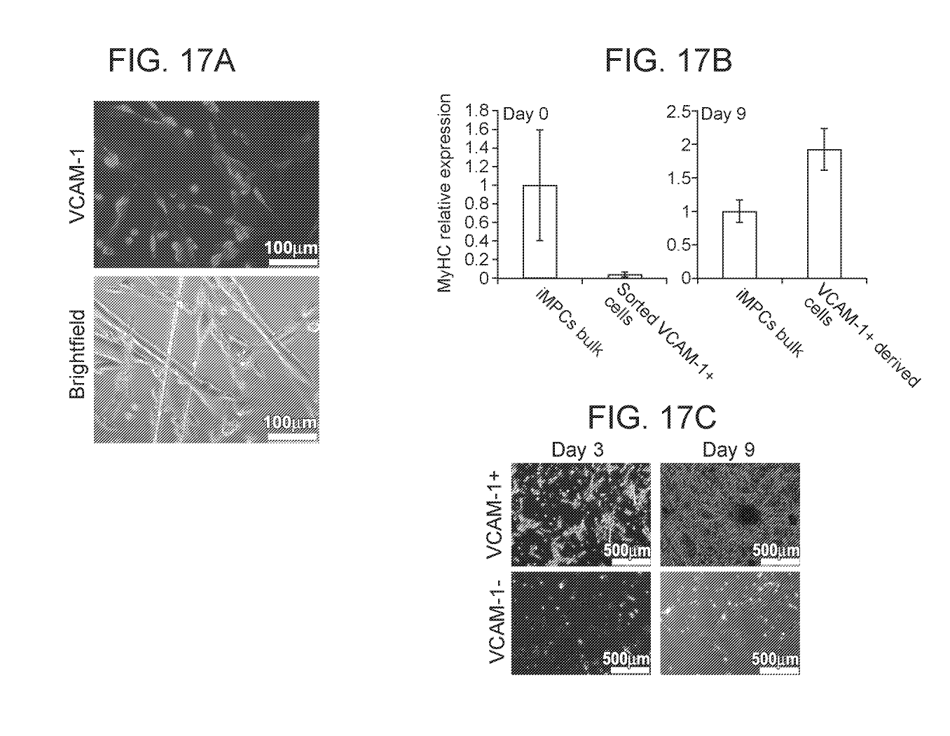

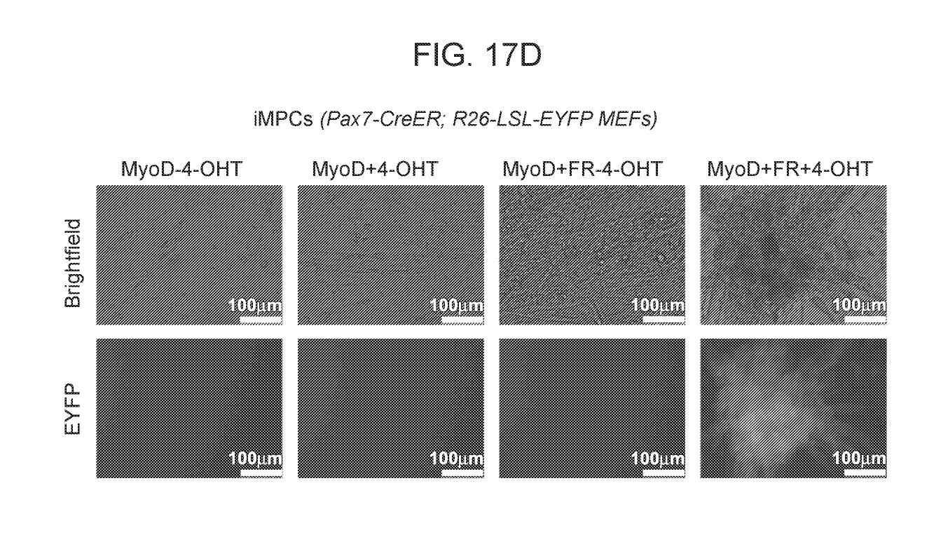

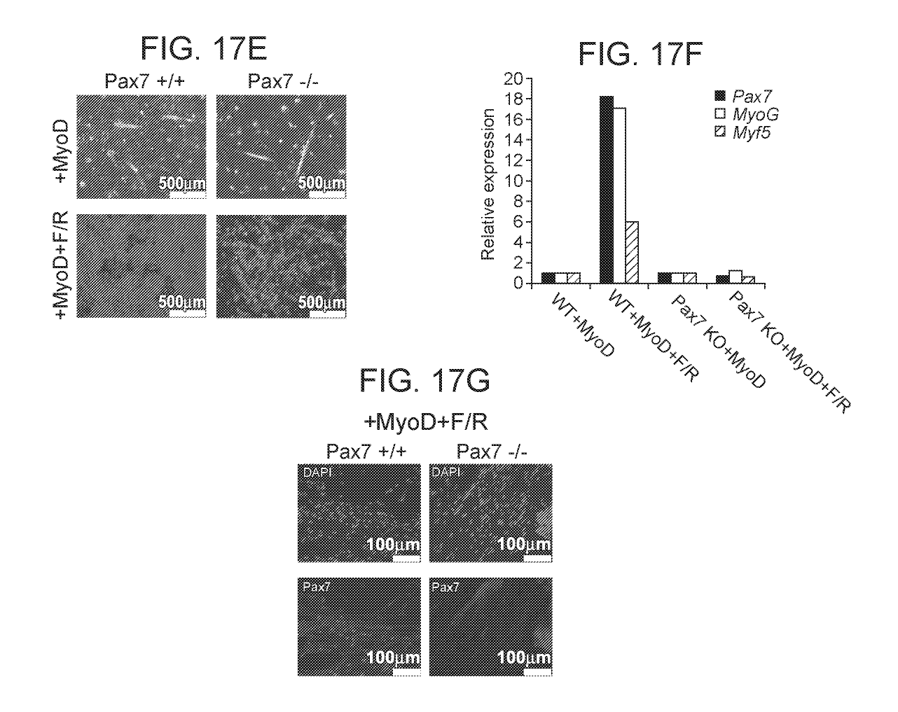

Without being bound by any particular theory, the data suggest that iMPC cultures contain myogenic cells with molecular and functional characteristics of stem, progenitor and differentiated cells, raising the question of whether iMPC generation and maintenance recapitulate stages of normal myogenesis. To determine a possible lineage hierarchy within iMPC subsets, expression of the surface marker VCAM1, which has recently been associated with both quiescent and activated satellite cells, was examined. The majority of mononucleated iMPCs were positive for VCAM1 expression were observed using flow cytometric analysis, consistent with a satellite cell or myoblast identity (FIG. 6G). Accordingly, immunofluorescence analysis showed that myofibers were negative while mononucleated cells were positive for VCAM1 expression within heterogeneous iMPC cultures (FIG. 17A). Critically, purified VCAM1⁺ iMPCs initially lacked MyHC expression by RT-qPCR, yet upregulated MyHC expression and initiated proliferative and contractile colonies upon culturing for 9 days. VCAM1− iMPCs did not give rise to proliferating cells or contractile myotubes, consistent with a differentiated phenotype (FIGS. 17B and 17C). These results point to a differentiation hierarchy between undifferentiated VCAM1⁺ stem/progenitor-like cells and VCAM1− differentiated progeny akin to myogenic cells in vivo.

To further explore the hierarchical relationship among iMPC subsets and their possible resemblance to satellite cells, iMPCs from MEFs carrying a satellite cell-specific Pax7-CreER allele9 as well as a ROSA26-LSLEYFP reporter were generated (FIG. 6A). EYFP⁺ cells were not detected in MEFs exposed to 4-OHT, ruling out the presence of contaminating Pax7⁺ myogenic or neural cells (FIG. 6B). Moreover, expression of MyoD alone in these MEFs was insufficient to activate the reporter in the presence of 4-OHT (FIG. 6D). By contrast, induction of MyoD in either MEFs or TTFs in the presence of bFGF and F/R activated the EYFP reporter in 2-3% of cells after 6 days of 4-OHT treatment (FIG. 6D and FIG. 6F) and this fraction progressively increased to 69% after 3 passages when using MEFs (FIG. 6H). The EYFP signal was observed not only in mononucleated cells but also in polynucleated myofibers 1-2 weeks after 4-OHT treatment (FIG. 17D and FIG. 6E). These assays demonstrate that iMPC cultures contain undifferentiated myogenic cells with satellite cell characteristics, which expand and differentiate into mature, contracting myotubes upon further passaging.

iMPC Maintenance Requires the Satellite Cell Master Regulator Pax7

Considering that a subset of undifferentiated iMPCs expresses Pax7, which is essential for satellite cells, the establishment or maintenance of iMPC cultures was tested to see if it is dependent upon Pax7 function. Pax7^{+/-} mice were intercrossed to obtain both Pax7^{-/-} experimental and Pax7^{+/+} control MEFs, which were infected with lentiviral vectors expressing M2rtTA and tet-OP-MyoD and subsequently exposed to either transdifferentiation (MyoD) or reprogramming (MyoD+F/R) conditions. MyoD expression alone yielded polynucleated myotubes from both Pax7^{+/+} and Pax7^{-/-} MEFs, indicating that Pax7 is dispensable for the direct conversion of fibroblasts to myotubes (FIG. 17E). Moreover, proliferative and contractile colonies were detected in both Pax7^{+/+} or Pax7^{-/-} cultures upon overexpression of MyoD and treatment with dox and F/R, suggesting that Pax7 is also dispensable for the establishment of iMPC-like cells (data not shown). However, the Pax7^{-/-} iMPC-like cultures were not able to be maintained despite the presence of F/R. Specifically, Pax7^{-/-} cultures ceased to proliferate and contract over time, leaving behind only post-mitotic myotubes that lacked Pax7 or Myf5 expression (FIG. 17E-17G). Without being bound by any particular theory, the data suggest that iMPC propagation in vitro relies on the same genetic program as satellite cell maintenance in vivo, providing mechanistic evidence that these two cell states are related.

Derivation of iMPCs from Muscle and MEFs Using Small Molecules Alone

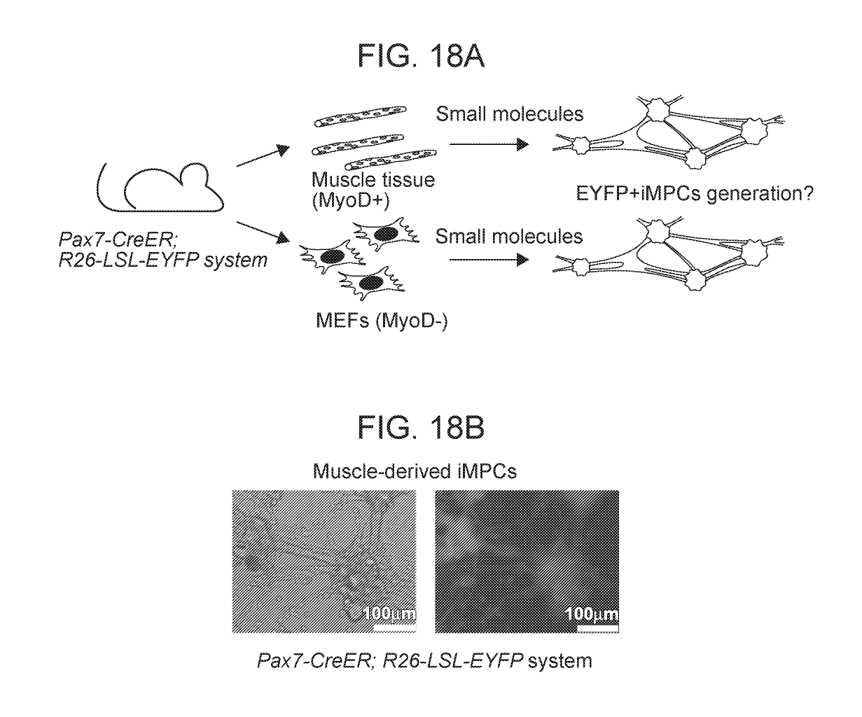

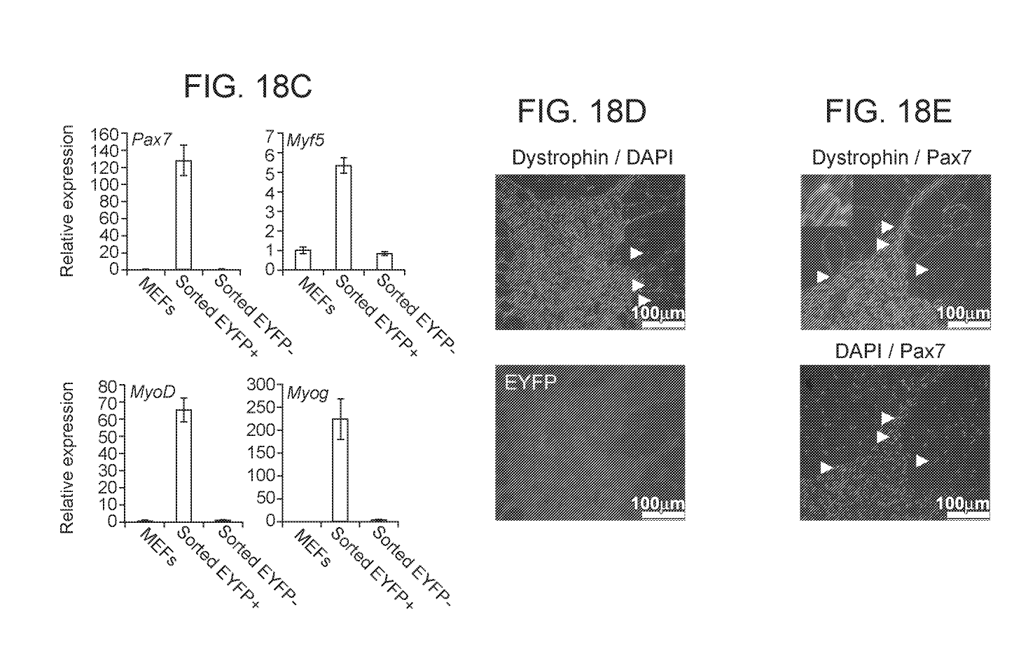

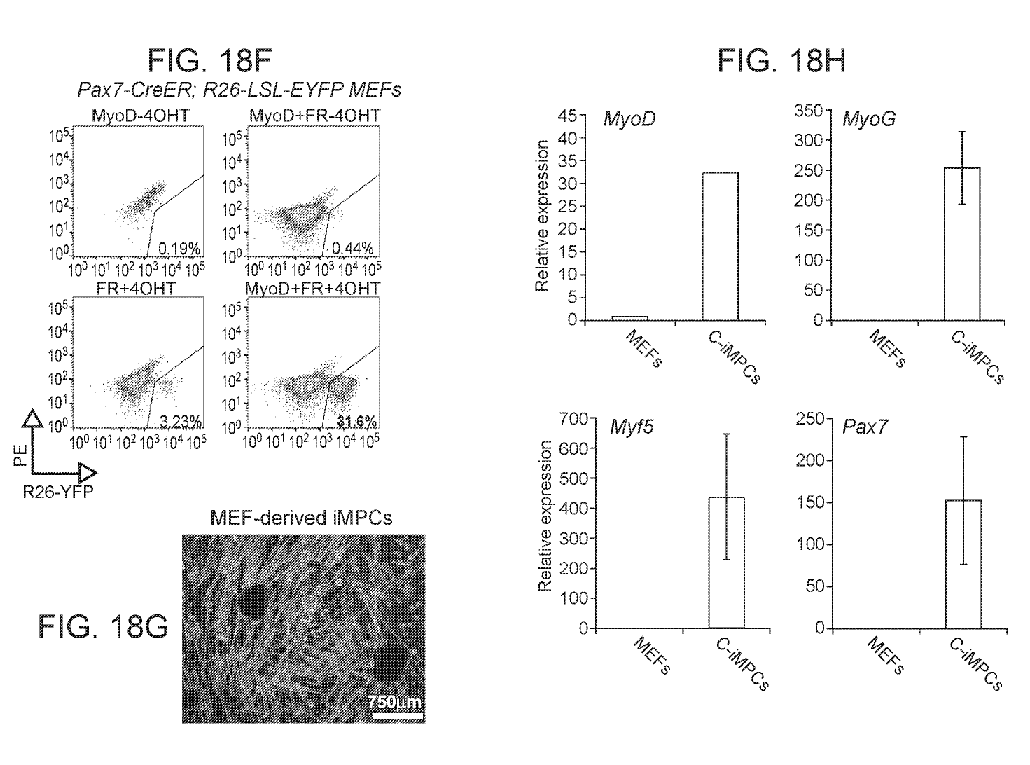

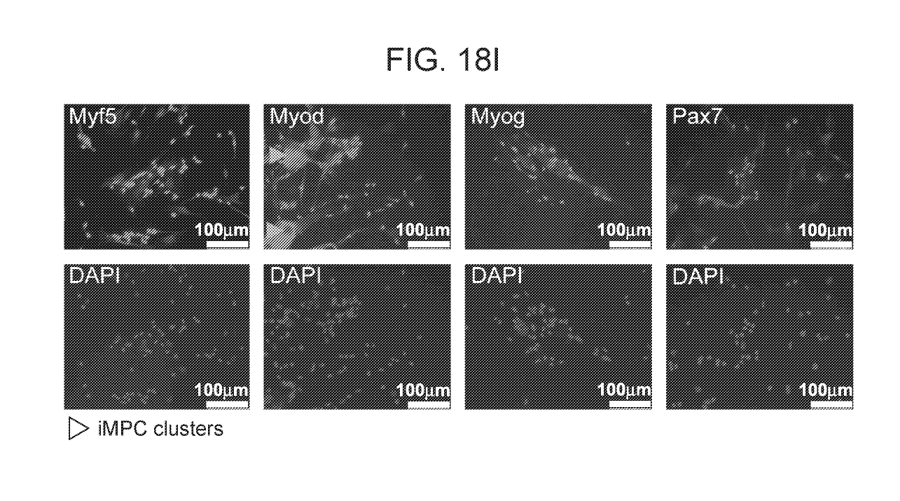

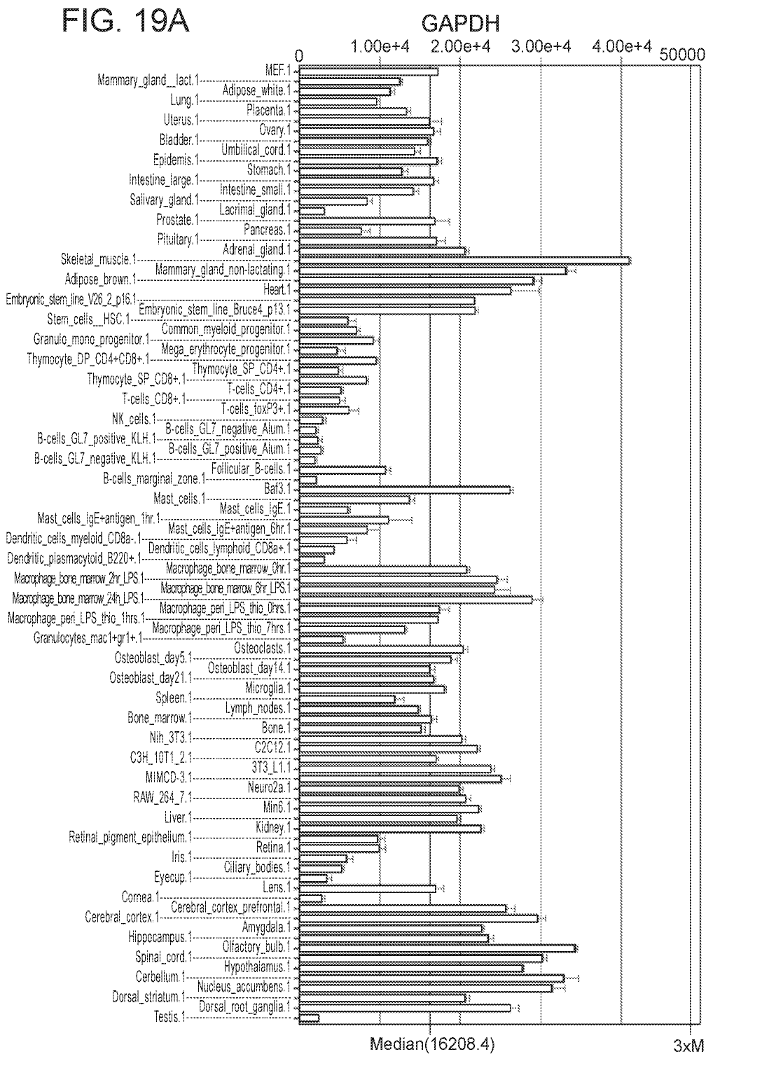

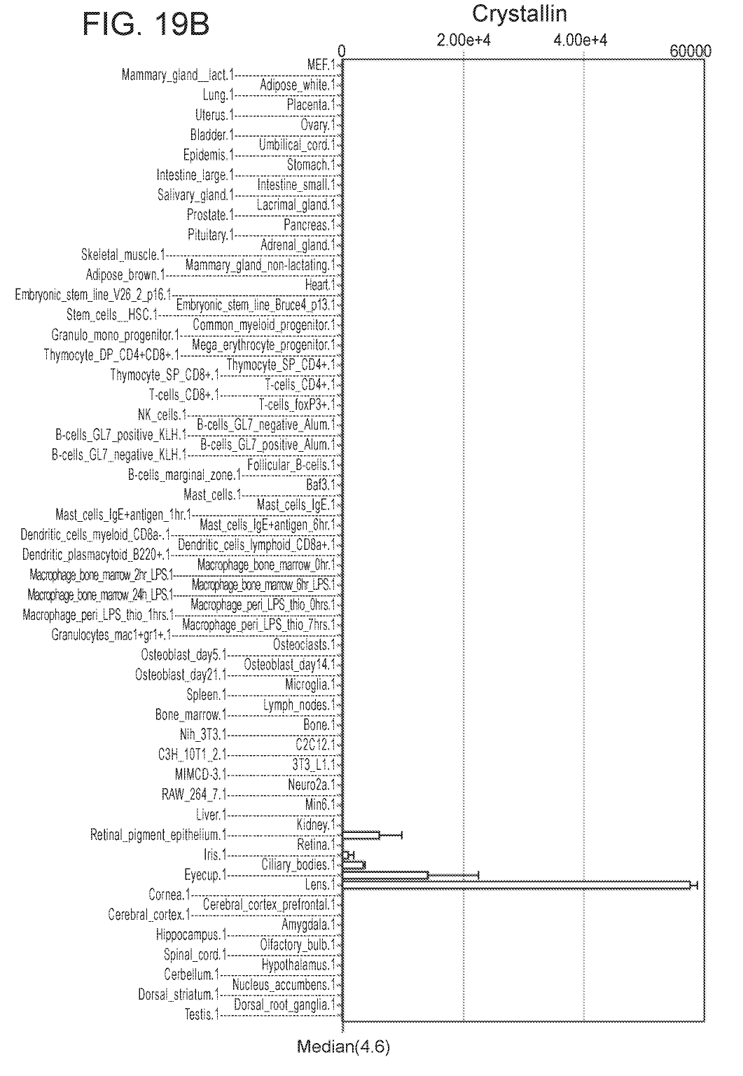

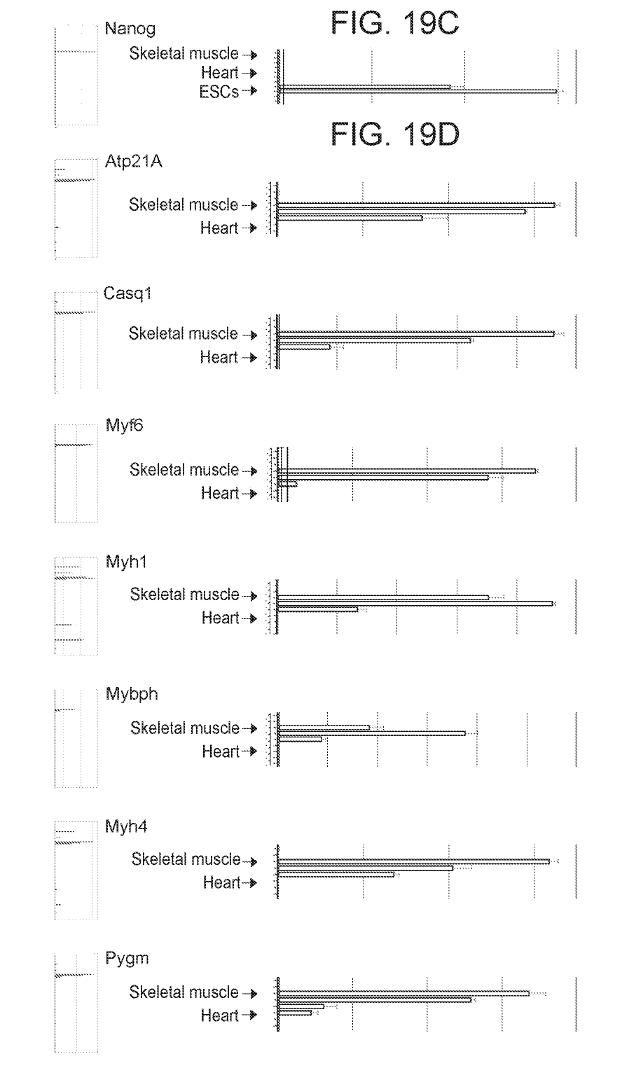

The observation that exogenous MyoD expression and small molecule treatment endows fibroblasts with a myogenic progenitor cell state raises the question of whether small molecules alone are sufficient to capture an iMPC-like state in primary muscle cells that already express endogenous MyoD. To test this hypothesis, muscle tissue from Pax7-CreER; ROSA26-LSL-EYFP mice was explanted, mononuclear cells were isolated through mechanical and enzymatic digestion and cultured in iMPC medium (DMEM, KOSR, FBS, bFGF, F/R) (FIG. 18A, top row). Indeed, iMPC-like colonies that activated the EYFP reporter upon 4-OHT treatment were established (FIG. 18B) and could be propagated for several passages (data not shown). Consistent with this finding, RT-qPCR showed that sorted EYFP⁺ cells expressed myogenic stem, progenitor and differentiation genes compared to EYFP⁻ cells 14 days after 4-OHT treatment (FIG. 18C). Myoblast-derived iMPC-like cells engrafted and differentiated into Dystrophin⁺ myofibers with centrally located nuclei and EYFP fluorescence following transplantation into mdx mice, documenting their differentiation and regeneration potential in vivo (FIG. 18D). These muscle-derived iMPCs are referred to as M-iMPCs to distinguish them from fibroblast-derived iMPCs. Notably, rare Pax7⁺ cells within Dystrophin⁺ areas were detected. Without being bound by any particular theory, this suggests that transplanted M-iMPCs may replenish the endogenous satellite cell pool (FIG. 18E).

Given that treatment of fibroblasts with demethylating compounds results in the desilencing of the endogenous MyoD locus, the inventors tested whether prolonged exposure of MEFs to our small molecules—some of which have previously been associated with genomic demethylation—may generate iMPCs in the absence of exogenous MyoD expression (FIG. 18A, bottom row). Pax7-CreER, ROSA26-LSL-EYFP MEFs were treated with bFGF, F/R and ascorbate for up to three weeks in the presence or absence of exogenous MyoD expression, followed by flow analysis for EYFP⁺ cells. Remarkably, a rare population of EYFP⁺ cells (3.2%) were detected after 18 days of treatment with small molecules and this fraction further increased to 32% when MyoD was simultaneously overexpressed (FIG. 18F). Consistent with this result, the emergence of colonies and contractile myotubes expressing Pax7, Myf5, Myog and MyoD was detected by RT-qPCR or immunofluorescence; these myogenic colonies are referred to as chemically induced iMPCs (C-iMPCs) (FIG. 18G-18I). It can be concluded that iMPC culture conditions are sufficient to derive and maintain myogenic stem/progenitor cells from muscle and MEFs, albeit at a significantly lower efficiency and with delayed kinetics. These results also indicate that exogenous MyoD expression assists in, but is not essential for reprogramming.

### Discussion

It has been notoriously difficult to culture primary myogenic cell populations for extended periods of time without losing proliferation and transplantation potential. Here, the inventors provide evidence that transient MyoD induction in fibroblasts, combined with small molecule treatment, readily induces a myogenic progenitor cell state, which shares characteristics with satellite cells. This includes the activation of the endogenous Pax7 locus, the requirement for Pax7 itself to self-renew and the potential to differentiate into functional myofibers in vitro and in vivo. Importantly, the culture conditions not only enable reprogramming of fibroblasts into iMPCs but also facilitate permanent capture of myogenic stem/progenitor cells from muscle tissue. This study is the first to report on a stable cell culture model of non-transformed myogenic cells with molecular and functional properties of muscle stem/progenitor cells. It remains to be determined whether purified Pax7⁺ iMPCs are transcriptionally and functionally equivalent to muscle-derived Pax7⁺ cells and understanding why mononucleated iMPCs assemble into heterogeneous cultures containing both stem/progenitor cells as well as differentiated myotubes. Without being bound by any particular theory, it is possible that myotubes could provide physical or chemical support for parental myogenic progenitors in vitro. Recent data suggest that mature myofibers recreate a niche in vitro by secreting signals that maintain satellite cells in a quiescent state. Thus, without being bound by any particular theory, it may thus be possible to generate more homogeneous stem and progenitor cell cultures by supplementing the heterogeneous iMPC culture system with additional compounds that enhance satellite cell expansion, such as p38 inhibitors.

MyoD has been mostly studied as a pro-differentiation factor in the context of myogenesis or transdifferentiation. Without being bound by any particular theory, this data suggests that MyoD also functions as a de-differentiation factor in the presence of appropriate signals. The inventors surmise that the concomitant expression of MyoD and exposure to small molecules enables both the capture of this transient myoblast like state and the dedifferentiation towards a Pax7⁺ stem-like state. Without being bound by any particular theory, mechanistically, the inventors hypothesize that F/R and ascorbic acid facilitate down-regulation of the fibroblast program and desilencing of genes associated with muscle stem and progenitor cells. Once the endogenous MyoD, Myf5 and Pax7 loci have been activated in iMPCs, these small molecules may be required to stabilize and maintain a self-renewing stem/progenitor cell state. Of note, these compounds also appear to promote terminal differentiation and maturation of myogenic stem/progenitor-like cells based on the finding described herein, that myofibers spontaneously contract and express markers associated with adult muscle (e.g. Myh6, Car3, Casq1, Mstn), which was never observed during MyoD mediated transdifferentiation.

In addition to providing mechanistic insights and a useful tool to study the role of transcription factors and external stimuli in cell fate control, the data described herein can have therapeutic implications. For example, patient-specific iMPCs might be useful for the study of myogenic disorders ex vivo as well as for small molecule screens that reverse disease phenotypes. Similarly, iMPCs derived from Duchenne muscular dystrophy patients can in principle be used for cell therapy following restoration of Dystrophin expression using CRISPR-Cas9 technology. Lastly, the observation that myotubes expressing adult-muscle markers and displaying vigorous contractions are present, in iMPCs, may provide a valuable source for tissue engineering purposes.

### Example 3

### Materials and Methods

Construction of the tetOP-MyoD Plasmid

A doxycycline-inducible MyoD lentivirus was generated by excising the mouse Myodl gene from the CMV-myoD expression vector using EcoRI (addgene, plasmid #8398). The 1785 bp fragment was inserted into the EcoRI site of the pLV-tetO backbone (addgene, plasmid #19765). The correct orientation was verified by sequencing.

### Animals

The following mouse strains were obtained from Jackson Laboratories: (i) B10ScSn.Cg-Prkdcscid Dmdmdx/J, stock number 018018, (ii) C57BL/10ScSn-Dmdmdx/J, stock number 001801, (iii) B6.Cg-Pax7tml(cre/ERT2)Gaka/J, stock number 017763, (iv) B6 (SJL)-Pou5fltm1.1(cre/Esr1*)Yseg/J, stock number 016829 and (v) B6.129X1-Gt(ROSA)26Sortm 1(EYFP)Cos/J, stock number 00618. All procedures, including maintenance of animals, were performed in compliance with an active IACUC protocol and according to guidelines of the MGH Subcommittee on Animal Research Care.

### Cell Culture

Mouse embryonic fibroblasts (MEFs), Tail-tip fibroblasts (TTFs) and the commercial myoblast cell line C2C12 (ATCC®CRL-1772™) were cultured in “MEF medium” containing DMEM (ThermoFisher Scientific, catalog number 10313-021), supplemented with 10% Fetal Bovine Serum (FBS) (HyClone catalog number SH30396.03), 1% GlutaMAX (ThermoFisher Scientific, catalog number 35050061), 1% non-essential amino acids (ThermoFisher Scientific, catalog number 11140050), 1% penicillin-streptomycin (ThermoFisher Scientific, catalog number 15140122), 0.5% (3-mercaptoethanol (ThermoFisher Scientific, catalog number 21985-023). Freshly isolated satellite cells and derivative myoblasts were cultured using a 1:1 ratio of DMEM and F-10 (1×) Nutrient mix (ThermoFisher Scientific, catalog number 11550-043) supplemented with 10% horse serum (ThermoFisher Scientific, catalog number 16050-122), 20% FBS (HyClone catalog number SH30396.03) and 10 ng/ml basic-FGF (R&D 233-FB). Satellite cells and myoblasts were cultured on plates coated with Matrigel Basement Membrane Matrix (Catalog number 356237, Corning). Reprogramming of MEFs and TTFs into iMPCs Reprogramming of MEFs or TTFs into iMPCs was performed using “iMPC medium” containing KnockOut-DMEM (ThermoFisher Scientific, catalog number 10829-018) supplemented with 10% FBS (HyClone catalog number SH30396.03), 10% KnockOut Serum Replacement (ThermoFisher Scientific, Catalog number 10828028) 1% GlutaMAX (Catalog number 35050061), 1% non-essential amino acids (ThermoFisher Scientific, Catalog number 11140050), 1% penicillin-streptomycin (ThermoFisher Scientific, catalog number 15140122), 0.5% 0-mercaptoethanol (ThermoFisher Scientific, catalog number 21985-023) and 10 ng/ml basic FGF (R&D 233-FB). Forskolin (Sigma-Aldrich F6886) and RepSox (Sigma-Aldrich, R0158) were added at a concentration of 5 μM to induce iMPC formation. For some experiments 3μM of the GSK3β inhibitor CHIR99021 (Tocris) was used. Doxycycline (Sigma-Aldrich, D9891) was added at a concentration of 2 ug/ml. For all reprogramming experiments, cells were reprogrammed in “iMPC medium” with and without Forskolin and RepSox. Expanded bulk cultures or picked iMPC clones were cultured in iMPC medium supplemented with Forskolin and RepSox at a concentration of 5 μM without dox. To assess the contribution of ascorbic acid to iMPC formation, cells were cultured in “iMPC medium” without Serum Replacement and supplemented with ascorbic acid (50 ug/ml), and Forskolin and RepSox at a final concentration of 5 μM.

Generation of Pax7-CreER; Rosa26-loxSTOPlox-EYFP MEFs and iMPCs

Pax7-CreER mice, termed B6.Cg-Pax7tml(cre/ERT2)Gaka/J were purchased from Jackson Laboratory (stock number 017763). Pax7-CreER mice were crossed with ROSA26-lox-STOP-lox-EYFP mice to produce bitransgenic reporter MEFs or myoblasts. Genotyping of the Pax7-CreER allele was performed as previously described (Murphy et al., Satellite cells, connective tissue fibroblasts and their interactions are crucial for muscle regeneration. Development (2011) 138, 3625-3637). Cells with the correct genotype were infected with lentiviral vectors harboring M2rtTA and tetOP-MyoD alleles and reprogrammed in either iMPC medium with and without F/R. 4-hydroxytamoxifen (4-OHT) (Sigma-Aldrich, H7904) was used at a concentration of 100 nM for all subsequent experiments and was continuously added to the culture medium to induce labeling. For in vivo labeling of satellite cells, 1 mg tamoxifen (Sigma-Aldrich, T5648) was diluted in 10 mg/ml corn oil (Sigma-Aldrich, C8267) and injected into the peritoneum of a Pax7-CreER; ROSA26-lox-STOP-lox-EYFP mice on 3 consecutive days.

### Viral Vector Production

For lentiviral supernatant generation, confluent (˜90%) T-293 cells in 10 cm culture dish plates were transfected with a solution consisting of 770 μl Opti-MEM (Gibco) and 50 μl of TransIT-LT1 (Mirus), A8.9 (8.5 μg), VSV-G (5.5 μg) and 11 μg of the target plasmid (M2rtTA or tetOP-MyoD). Cells were transfected using regular MEF medium without penicillin-streptomycin. Twenty-four hours after transfection, the medium was replaced, and 48 and 72 hrs after transfection the supernatant was collected, filtered through a 0.45-μM filter (Westnet), supplemented with 4-8 μg/ml polybrene (Sigma-Aldrich) and added freshly to the cells. Similar ratios of M2rtTA and tetOP-MyoD1 (1:1) were used.

### Quantitative RT-PCR Analysis

DNase-treated total RNA was extracted using RNeasy Mini Kit (Qiagen) according to the manufacturers' instructions. cDNA was generated using the Transcriptor First-Strand cDNA Synthesis Kit (Roche, 04379012001). Quantitative RT-PCR was carried out using the Brilliant Master Mix (Agilent Technologies). Relative expression was calculated using GAPDH as a house keeping gene.

TABLE 1
qRT-PCR Primers

| | | SEQ ID |
|---|---|---|
| Primer Name | Primer Sequence | NO |
| GAPDH-forward | TGGTATCGTGGAAGGACTCA | 1 |
| GAPDH-reverse | TTCAGCTCAGGGATGACCTT | 2 |
| MyoG-Forward | GAGACATCCCCCTATTTCTACCA | 3 |
| MyoG-Reverse | GCTCAGTCCGCTCATAGCC | 4 |
| MyHC-forward | TTCATTGGGGTCTTGGACAT | 5 |
| MyHC-reverse | AACGTCCACTCAATGCCTTC | 6 |
| Myf5-forward | GCCTGAAGAAGGTCAACCAG | 7 |
| Myf5-reverse | CCATCAGAGCAGTTGGAGGT | 8 |
| Pax7-forward | GTGGAATCAGAACCCGACCTC | 9 |
| Pax7-reverse | GTAGTGGGTCCTCTCAAAGGC | 10 |
| MyoD Forwrad | TCGACACAGCCGCACTCTTC | 11 |
| MyoD Reverse | CACTACAGTGGGGACTCAGATGC | 12 |

### Immunocytochemistry

For immunocytochemistry, cultured cells were first washed with PBS, cross linked with 4% paraformaldehyde (PFA) (EMS, 15710) for 5 minutes, washed with PBS and blocked for ½ hr at room temperature (RT). The blocking solution consisted of 2% BSA dissolved in PBS and 0.1% Triton-X-100. Primary antibodies were diluted in blocking solution and incubated for 1 hr at RT. The primary antibodies used in this study were: Rabbit anti-MyoD1 (Sc-760, Santa Cruz, 1:200), Mouse IgG anti-Myogenin (sc-17320, Santa Cruz, 1:200), Mouse IgG1 anti-Pax7 (Clone Pax7, MAB1675, R&D 5 ug/ml), Mouse IgG2B anti-Myosin HC (R&D 1:500 clone MF20, MAB4479), and Rabbit IgG anti-Myf5 (Sc-302, Santa Cruz, C-20). The secondary antibodies used in this study were: A21202 Alexa Fluor 488 donkey anti-mouse IgG, A21141 Alexa Fluor 488 goat anti-mouse IgG2B, A11056 Alexa Fluor 546 donkey anti-goat IgG, A21123 Alexa Fluor 546 goat anti-mouse IgG1, A10040 Alexa Fluor donkey anti-rabbit IgG and A111055, Alexa Fluor 488 donkey anti-goat IgG, all at a 1:400 dilution. DAPI was used for nuclear counterstaining.

For immunocytochemistry on muscle tissue, slides containing muscle sections were cross-linked with 4% PFA, washed with PBS, incubated for ½ hr with 2% BSA dissolved in PBS and 0.1% Triton-X-100, followed by ½ hr incubation with 10% donkey serum (Sigma-Aldrich, D9663), and 10% rabbit serum diluted in PBS. Cells were then incubated for 1 hr in primary antibody at RT followed by 1×PBS rinse (×2) and incubation in secondary antibodies (1 hr at RT). Primary antibodies used were Rabbit anti-Dystrophin (ab15277, Abcam, 1:200) and chicken anti-EYFP/GFP (GFP-1020, AVES. 1:300). Secondary antibodies used were: A10040 Alexa Fluor 546 donkey anti-rabbit IgG and A11039 Alexa Fluor 488 goat anti-chicken IgG, both at 1:400 dilution.

### Live Antibody Staining and Flow Cytometry Analysis

For live staining of cells, PE-conjugated anti-mouse VCAM-1 (CD106) antibody (eBioscience, clone 429, catalog number 12-1061-80) was added directly to the cells. Cells were then incubated at 37° C. for 1 h, washed twice with 1×PBS and visualized for surface marker expression. For flow cytometric analysis, iMPC clones were harvested and stained with antibodies to Thy1 (eBioscience, clone 53-2.1, catalog number 48-0902-80), CD45 (eBioscience, clone 30-F11, catalog number 56-0451-83), VCAM-1 (eBioscience, clone 429, catalog number 12-1061-80), CD31 eBioscience, clone 390, catalog number 25-0311-82), and Sca1 (eBioscience, clone D7, catalog number 108129) for 1 hr at room temperature, washed, filtered and sorted using fluorescence-activated cell sorting (FACS) on an Aria II sorter (BD).

### RNA Extraction and Microarrays

DNase-treated total RNA was extracted using RNeasy Mini Kit (Qiagen) according to the manufacturers' instructions. Hybridization to the GeneChip Mouse 2.0 ST arrays (Affymetrix) was performed at the Partners Center for Personalized Genetic Medicine. RMA (robust multi-array average) was performed using Expression Console (Affymetrix). Scatter plots, analysis of linear regression coefficients and TTEST analysis were performed using Excel. Classification and annotations of up-regulated genes in iMPCs was performed using the DAVID online functional annotation tool (http://david.abcc.ncifcrf.gov/). The microarray data has been deposited in NCBI's Gene Expression Omnibus (GEO, accession number: GSE92336). Gene expression of activated satellite cells (ASCs) and Quiescent satellite cells (QSCs) was previously published and downloaded from GEO (GSE4717753), as well as for MEFs used in comparison (GSE6746254). For Venn diagram the online software Venny 2.1 was used (http://bioinfogp.cnb.csic.es/tools/venny/).

Intramuscular Transplantation of Myoblasts and iMPCs

Myogenic cell transplantation was performed as recently described (Gerli et al., Transplantation of induced pluripotent stem cell-derived mesoangioblast-like myogenic progenitors in mouse models of muscle regeneration. Journal of visualized experiments: JoVE, (2014), e50532). Briefly, target cells were detached by trypsinization, counted and centrifuged at 232×g for 5 minutes. The cell pellets have been washed twice in Ca⁺⁺/Mg⁺⁺-free PBS (Life Technologies, 14190-136) to remove residual xenogenic proteins present in the culture media. Cells were resuspended for injection in 1×PBS (Life Technologies, 14190-136) to a final concentration of 1×10⁶ cells/30 μl. The cell suspension was injected into the tibialis anterior muscle using 29 g insulin syringes (Exel int., catalog number 2628). The needle was inserted craniocaudally 2-5 mm into the muscles, with a 150 inclination relative to the tibia. The cell suspension was slowly released into the muscle while retracting the needle to allow for homogeneous dispersion and to limit cell spilling through the needle track. Grafts were harvested for sectioning 2-4 weeks post transplantation.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method for generating induced muscle progenitor cells (iMPCs), the method comprising: treating a population of somatic cells obtained from a subject with a cyclic AMP agonist, and a TGF-β inhibitor for a time and under conditions that induce dedifferentiation of the somatic cells to a population of cells comprising iMPCs, wherein the iMPCs are proliferative, self-renewing and capable of forming skeletal muscle myotubes.

2. The method of claim 1, wherein the somatic cells are fibroblasts.

3.-11. (canceled)

12. The method of claim 1, wherein the somatic cells are muscle biopsy or muscle-derived explants and the iMPCs are muscle-induced iMPCs (M-iMPCs).

13. The method of claim 1, further comprising culturing the somatic cells and/or population of cells comprising iMPCs with ascorbic acid or a GSK3β inhibitor.

14.-17. (canceled)

18. The method of claim 1, further comprising a step of isolating an iMPC and plating it as a clonal culture.

19. (canceled)

20. The method of claim 1, wherein the iMPCs can be maintained in culture for at least 4 months.

21. (canceled)

22. The method of claim 1, wherein the resulting cells do not comprise exogenous nucleic acid relative to the population of somatic cells.

23. (canceled)

24. (canceled)

25. The method of claim 1, wherein the dedifferentiation of the somatic cells to iMPCs does not go through a transient pluripotent state.

26. (canceled)

27. The method of claim 26, wherein the iMPCs do not detectably express fibroblast markers.

28. (canceled)

29. (canceled)

30. An in vitro heterogeneous population of skeletal muscle cells comprising induced muscle progenitor cells (iMPCs).

31. The population of claim 30, wherein the iMPCs do not comprise exogenous nucleic acid encoding a MyoD transcription factor.

32. The in vitro heterogeneous population of skeletal muscle cells of claim 30, wherein the heterogeneous population can be maintained in culture without loss of phenotype for at least 6 months.

33.-43. (canceled)

44. A method for promoting muscle regeneration and/or repair, the method comprising: administering a therapeutically effective amount of iMPCs to a subject in need thereof.

45. The method of claim 44, wherein the iMPCs are prepared according to the method of claim 1.

46. The method of claim 44, wherein the iMPCs are autologous to the subject.

47. (canceled)

48. (canceled)

49. A method for treating a muscle disease or disorder, the method comprising: administering a therapeutically effective amount of iMPCs to a subject in need thereof.

50. The method of claim 49, wherein the iMPCs are prepared according to the method of claim 1.

51. The method of claim 49, wherein the iMPCs are autologous to the subject.

52. (canceled)

53. (canceled)

54. The method of claim 49, wherein the muscle disease or disorder is characterized by a gene mutation and/or deficiency of a gene product.

55.-79. (canceled)

80. The method of claim 1, wherein the somatic cells are obtained from a subject having a muscular disease.

81. The method of claim 1, wherein the iMPCs are genetically modified to express a transgene.


SEQUENCE LISTING (12 sequences)

SEQ ID NO: 1 - DNA, 20 bp, Artificial Sequence
  Synthetic construct
tggtatcgtggaaggactca

SEQ ID NO: 2 - DNA, 20 bp, Artificial Sequence
  Synthetic construct
ttcagctcagggatgacctt

SEQ ID NO: 3 - DNA, 23 bp, Artificial Sequence
  Synthetic construct
gagacatccccctatttctacca

SEQ ID NO: 4 - DNA, 19 bp, Artificial Sequence
  Synthetic construct
gctcagtccgctcatagcc

SEQ ID NO: 5 - DNA, 20 bp, Artificial Sequence
  Synthetic construct
ttcattggggtcttggacat

SEQ ID NO: 6 - DNA, 20 bp, Artificial Sequence
  Synthetic construct
aacgtccactcaatgccttc

SEQ ID NO: 7 - DNA, 20 bp, Artificial Sequence
  Synthetic construct
gcctgaagaaggtcaaccag

SEQ ID NO: 8 - DNA, 20 bp, Artificial Sequence
  Synthetic construct
ccatcagagcagttggaggt

SEQ ID NO: 9 - DNA, 21 bp, Artificial Sequence
  Synthetic construct
gtggaatcagaacccgacctc

SEQ ID NO: 10 - DNA, 21 bp, Artificial Sequence
  Synthetic construct
gtagtgggtcctctcaaaggc

SEQ ID NO: 11 - DNA, 20 bp, Artificial Sequence
  Synthetic construct
tcgacacagccgcactcttc

SEQ ID NO: 12 - DNA, 23 bp, Artificial Sequence
  Synthetic construct
cactacagtggggactcagatgc